(19) United States
(12) Patent Application Publication
(10) Pub. No.: US 2009/0005363 A1
(43) Pub. Date: Jan. 1, 2009

(54) Organic Compounds

(76) Inventors: Ralf Glatthar, Bad Sackingen (DE); David Orain, Hesingue (FR); Carsten Spanka, Lorrach (DE)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/158,387

(22) PCT Filed: Dec. 18, 2006
(86) PCT No.: PCT/EP2006/012181

§ 371 Date: Jun. 20, 2008

(30) Foreign Application Priority Data
Dec. 20, 2005 (EP) 05027934.8
Sep. 11, 2006 (EP) 06120424.4

(51) Int. Cl. A61K 31/55; C07D 211/68; C07D 413/02; C07D 291/06; C07D 401/06; A61P 1/00; A61P 13/02; C07D 403/02; A61K 31/4545; A61K 31/5375; A61K 31/546

(52) U.S. Cl. 514/217.05; 546/194; 544/124; 544/2; 540/597; 540/598; 514/318; 514/235.5; 514/222.5; 514/217.04

(57) ABSTRACT

The present invention relates to novel nicotinic acid derivatives, of formula (I)

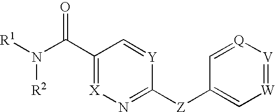

wherein the substituents are defined in the specification, their preparation, their use as pharmaceuticals and pharmaceutical compositions containing them.

The present invention relates to novel nicotinic acid derivatives, their preparation, their use as pharmaceuticals and pharmaceutical compositions containing them.

WO2005/079802 describes bipyridylamides and their use as modulators of metabotropic glutamate receptor-5. The compounds show valuable properties, but also have disadvantages. Thus, there is a need to provide further compounds having properties as modulators of metabotropic glutamate receptor-5.

In a first aspect, the invention relates to a compound of formula (I)

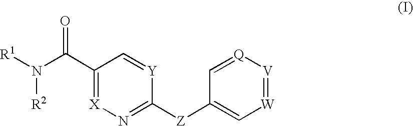

wherein

R¹ represents optionally substituted Alkyl or optionally substituted Benzyl and

R² represents Hydrogen (H), optionally substituted Alkyl or optionally substituted Benzyl; or

R¹ and R² form together with the Nitrogen atom to which they are attached an optionally substituted heterocycle with less than 14 ring atoms;

R³ represents Halogen, Hydroxy (OH), Alkyl, Alkoxy, Amino, Alkylamino, Dialkylamino;

R⁴ represents Hydroxy (OH), Halogen, Amino, Alkylamino, Dialkylamino Alkyl, Alkoxy;

Q represents CH, CR⁴, N;

V represents CH, CR⁴, N;

W represents CH, CR⁴, N;

X represents CH, N;

Y represents CH, CR³, N;

Z represents CR^{6a}R^{6b}, NR⁵, O;

R⁵ represents Hydrogen, Hydroxy (OH);

R^{6a} and R^{6b} are each independently selected from Hydrogen, Halogen, Hydroxy (OH), Amino, Alkyl, Alkoxy, Haloalkyl; and

provided that Q, V, W are not N at the same time in free base or acid addition salt form for use as a pharmaceutical.

In the present specification, the following definitions shall apply if no specific other definition is given:

“Alkyl” represents a straight-chain or branched-chain alkyl group, preferably represents a straight-chain or branched-chain C₁₋₁₂alkyl, particularly preferably represents a straight-chain or branched-chain C₁₋₆alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl, with particular preference given to methyl, ethyl, n-propyl and iso-propyl. Alkyl may be unsubstituted or substituted. Exemplary substituents include, but are not limited to hydroxyl, alkoxy, halogen and amino. An example of a substituted alkyl is trifluoromethyl.

“Alkanediyl” represents a straight-chain or branched-chain alkanediyl group bound by two different Carbon atoms to the molecule, it preferably represents a straight-chain or branched-chain C₁₋₁₂ alkanediyl, particularly preferably represents a straight-chain or branched-chain C₁₋₆ alkanediyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl.

Each alkyl part of “alkoxy”, “alkoxyalkyl”, “alkoxycarbonyl”, “alkoxycarbonylalkyl” and “halogenalkyl” shall have the same meaning as described in the above-mentioned definition of “alkyl”.

“Alkenyl” represents a straight-chain or branched-chain alkenyl group and may be substituted or unsubstituted, preferably C₂₋₆alkenyl, for example, vinyl, allyl, 1-propenyl, isopropenyl, 2-butenyl, 2-pentenyl, 2-hexenyl, etc. and preferably represents C₂₋₄ alkenyl.

“Alkenediyl” represents a straight-chain or branched-chain alkenediyl group bound by two different Carbon atoms to the molecule, it preferably represents a straight-chain or branched-chain C₂₋₆ alkanediyl; for example, —CH═CH—, —CH═C(CH₃)—, —CH═CH—CH₂—, —C(CH₃)═CH—CH₂—, —CH═C(CH₃)—CH₂—, —CH═CH—C(CH₃)H—, —CH═CH—CH═CH—, —C(CH₃)═CH—CH═CH—, —CH═C(CH₃)—CH═CH—, with particular preference given to —CH═CH—CH₂—, —CH═CH—CH═CH—. Alkenediyl may be substituted or unsubstituted

“Alkynyl” represents a straight-chain or branched-chain alkynyl group and may be substituted or unsubstituted, preferably C₂₋₆alkynyl, for example, ethenyl, propargyl, 1-propynyl, isopropenyl, 1-(2- or 3) butynyl, 1-(2- or 3) pentenyl, 1-(2- or 3) hexenyl, etc., preferably represents C₂₋₄alkynyl and particularly preferably represents ethynyl. Alkynyl may be substituted or unsubstituted.

“Aryl” represents an aromatic hydrocarbon group, preferably a C₆₋₁₀ aromatic hydrocarbon group; for example phenyl, naphthyl, especially phenyl. Aryl may be substituted or unsubstituted

“Aralkyl” denotes an “Aryl” bound to an “Alkyl” (both as defined above) an represents, for example benzyl, α-methylbenzyl, 2-phenylethyl, α,α-dimethylbenzyl, especially benzyl. Aralkyl may be substituted or unsubstituted

“Heterocycle” represents a saturated, partly saturated or aromatic ring system containing at least one hetero atom. Preferably, heterocycles consist of 3 to 11 ring atoms of which 1-3 ring atoms are hetero atoms. Heterocycles may be present as a single ring system or as bicyclic or tricyclic ring systems; preferably as single ring system or as benz-annelated ring system. Bicyclic or tricyclic ring systems may be formed by annelation of two or more rings, by a bridging atom, e.g. Oxygen, sulfur, nitrogen or by a bridging group, e.g. alkanediyl or alkenediyl or be connected by a direct bond. A Heterocycle may be substituted by one or more substituents selected from the group consisting of Oxo (═O), Halogen, Nitro, Cyano, Alkyl, Alkanediyl, Alkenediyl, Alkoxy, Alkoxyalkyl, Alkoxycarbonyl, Alkoxycarbonylalkyl, Halogenalkyl, Aryl, Aryloxy, Arylalkyl. Examples of heterocyclic moieties are: pyrrole, pyrroline, pyrrolidine, pyrazole, pyrazoline, pyrazolidine, imidazole, imidazoline, imidazolidine, triazole, triazoline, triazolidine, tetrazole, furane, dihydrofuran, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazolidine, isothiazole, isothiazoline, isothiazolidine, thiadiazole, thiadiazoline, thiadiazolidine, pyridin, piperidine, pyridazine, pyrazine, piperazine, triazine, pyrane, tetrahydropyrane, thiopyrane, tetrahydrothiopyran, oxazine, thiazine, dioxin, morpholine, purine, pterine, and the corresponding benz-annelated heterocycles, e.g. indole, isoindole, coumarine, cumaronecinoline, isochinoline, cinnoline and the like.

“Hetero atoms” are atoms other than Carbon and Hydrogen, preferably Nitrogen (N), Oxygen (O) or Sulfur (S).

“Halogen” represents Fluoro, Chloro, Bromo or Iodo, preferably represents Fluoro, Chloro or Bromo and particularly preferably represents Chloro.

“Substituted”, wherever used for a moiety, means that one or more hydrogen atoms in the respective moiety are replaced independently of each other by the corresponding number of substituents. Exemplary substituents include, but are not limited to hydroxyl, halogen, alkyl, alkoxy and amino.

Compounds of formulae (I), (II), (III) or (IV) may exist in free or acid addition salt form. In this specification, unless otherwise indicated, language such as “compounds of formula (I)”, for example is to be understood as embracing the compounds in any form, for example free base or acid addition salt form. Salts which are unsuitable for pharmaceutical uses but which can be employed, for example, for the isolation or purification of free compounds of formula (I), (II), (III) or (IV), such as picrates or perchlorates, are also included. For therapeutic use, only pharmaceutically acceptable salts or free compounds are employed (where applicable in the form of pharmaceutical preparations), and are therefore preferred.

Tautomers can, e.g., be present in cases where amino or hydroxy, each with a least one bound hydrogen, are bound to carbon atoms that are bound to adjacent atoms by double bonds (e.g. keto-enol or imine-enamine tautomerism).

On account of the asymmetrical carbon atom(s) that may be present in the compounds of formulae (I), (II), (III) or (IV) and their salts, the compounds may exist in optically active form or in form of mixtures of optical isomers, e.g. in form of racemic mixtures or diastereomeric mixtures. All optical isomers and their mixtures, including the racemic mixtures, are part of the present invention.

In a further aspect, the invention relates to new compounds of formula (II)

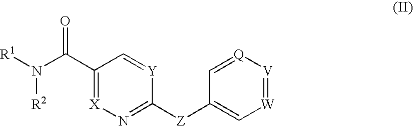

wherein

Q represents CH, CR⁴, N;

V represents CH, CR⁴, N;

W represents CH, CR⁴, N;

X represents CH, N;

Y represents CH, CR³, N;

Z represents CR^{6a}R^{6b}, NR⁵, O;

R¹ represents optionally substituted Alkyl or optionally substituted Benzyl and

R² represents Hydrogen (H), optionally substituted Alkyl or optionally substituted Benzyl; or

R¹ and R² form together with the Nitrogen atom to which they are attached an optionally substituted heterocycle with less than 14 ring atoms;

R³ represents Halogen, Hydroxy (OH), Alkyl, Alkoxy, Amino, Alkylamino, Dialkylamino;

R⁴ represents Hydroxy (OH), Halogen, Amino, Alkylamino, Dialkylamino Alkyl, Alkoxy;

R⁵ represents Hydrogen, Hydroxy (OH);

R^{6a} and R^{6b} are each independently selected from Hydrogen, Halogen, Hydroxy (OH), Amino, Alkyl, Alkoxy, Haloalkyl; and

provided that Q, V, W are not N at the same time and provided that at least one Q, V, W represents N, in free base or acid addition salt form.

In yet a further aspect, the invention relates to new compounds of formula (III)

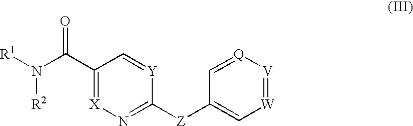

wherein

Q represents CH, CR⁴, N;

V represents CH, CR⁴, N;

W represents CH, CR⁴, N;

X represents CH, N;

Y represents CR³;

Z represents CR^{6a}R^{6b}, NR⁵, O;

R¹ represents optionally substituted Alkyl or optionally substituted Benzyl and

R² represents Hydrogen (H), optionally substituted Alkyl or optionally substituted Benzyl; or

R¹ and R² form together with the Nitrogen atom to which they are attached an optionally substituted heterocycle with less than 14 ring atoms;

R³ represents Halogen, Hydroxy (OH), Alkyl, Alkoxy, Amino, Alkylamino, Dialkylamino;

R⁴ represents Hydroxy (OH), Halogen, Amino, Alkylamino, Dialkylamino Alkyl, Alkoxy;

R⁵ represents Hydrogen, Hydroxy (OH);

R^{6a} and R^{6b} are each independently selected from Hydrogen, Halogen, Hydroxy (OH), Amino, Alkyl, Alkoxy, Haloalkyl; and

provided that Q, V, W are not N at the same time and provided that at least one Q, V, W represents N, in free base or acid addition salt form.

Preferred substituents, preferred ranges of numerical values or preferred ranges of the radicals present in the formula (I), (II) and (III) and the corresponding intermediate compounds are defined below.
X preferably represents CH.
Y preferably represents CH or CR³, wherein R³ preferably represents halogen, particular preferably chloro.

R^{6a} and R^{6b}, if present, preferably are both Hydrogen.

Z is preferably selected from NH, CH₂ and O.

Z preferably represents NH.

R³ preferably represents Halogen, Alkyl, Alkoxy, Alkylamino, Dialkylamino;

R³ more preferably represents Fluoro, Chloro, C₁₋₄ alkyl, e.g. methyl.

R³ particularly preferably represents chloro.

R⁴ preferably represents Hydroxy (OH), Halogen, Alkyl, Alkoxy.

R⁴ particularly preferably represents alkyl, e.g. methyl or Haloalkyl (substituted alkyl), e.g. trifluoromethyl.
R¹ and R² form together with the Nitrogen atom to which they are attached preferably represent an unsubstituted or substituted heterocycle having 3-11 ring atoms and 1-4 hetero atoms; the hetero atoms being selected from the group consisting of N, O, S, the substituents being selected from the group consisting of Oxo (═O), Hydroxy, Halogen, Amino, Nitro, Cyano, C₁₋₄ Alkyl, C₁₋₄ Alkoxy, C₁₋₄ Alkoxyalkyl, C₁₋₄ Alkoxycarbonyl, C₁₋₄ Alkoxycarbonylalkyl, C₁₋₄ Halogenalkyl, C₆₋₁₀ Aryl, Halogen-C₆₋₁₀ Aryl, C₆₋₁₀ Aryloxy, C₆₋₁₀-Aryl-C₁₋₄ alkyl.
R¹ and R² form together with the Nitrogen atom to which they are attached particularly preferably represent an unsubstituted, a single or twofold substituted heterocycle having 5-9 ring atoms and 1-3 hetero atoms; the hetero atoms being selected from the group consisting of N, O; the substituents being selected from the group consisting of Halogen, C₁₋₄ Alkyl.
R¹ and R² form together with the Nitrogen atom to which they are attached very particularly preferably represent an unsubstituted, a single or twofold substituted heterocycle selected from the group consisting of

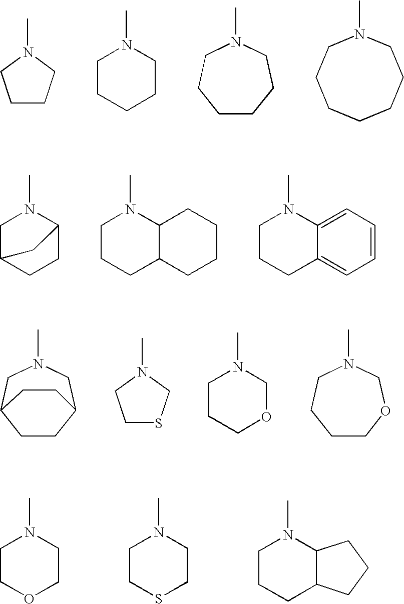

and the substituents being selected from the group consisting of halogen, e.g. fluoro, chloro; alkyl, e.g. methyl, ethyl, propyl, butyl; haloalkyl, e.g. trifluormethyl, fluoropropyl, difluoropropyl, e.g. 1,1-difluoropropyl or 1,2-difluoropropyl.
Where the heterocycle formed by R¹ and R² is substituted two fold or higher, the substituents may be on the same or different in-ring atoms.
R¹ and R² preferably represent, independent from each other, C₁-C₄ alkyl or benzyl, optionally substituted by C₁-C₄ alkoxy or halogen.

The above mentioned general or preferred radical definitions apply both to the end products of the formulae (I), (II), (III) and (IV) and also, correspondingly, to the starting materials or intermediates required in each case for the preparation. These radical definitions can be combined with one another at will, i.e. including combinations between the given preferred ranges. Further, individual definitions may not apply.

Preference according to the invention is given to compounds of the formulae (I), (II), (III) and (IV) which contain a combination of the meanings mentioned above as being preferred.

Particular preference according to the invention is given to compounds of the formulae (I), (II), (III) and (IV) which contain a combination of the meanings listed above as being particularly preferred.

Very particular preference according to the invention is given to the compounds of the formula (I), (II), (III) and (IV) which contain a combination of the meanings listed above as being very particularly preferred.

Preferred are compounds of formulae (I), (II) and (III) wherein R² represents an unsubstituted or substituted heterocycle.

Particular preferred are compounds of formulae (IIa to IId) as shown below:

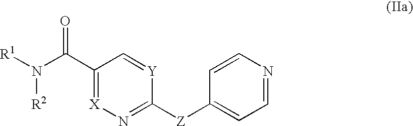

wherein the substituents have the meaning given in this specification.

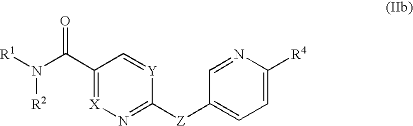

wherein the substituents have the meaning given in this specification.

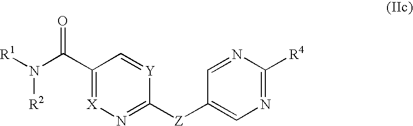

wherein the substituents have the meaning given in this specification.

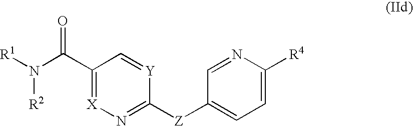

wherein R⁴ represents C₁-C₄alkyl, preferably methyl and the other substituents have the meaning given in this specification.

Further preferred compounds of the present invention have the formulae (IIIa to IIId) as shown below:

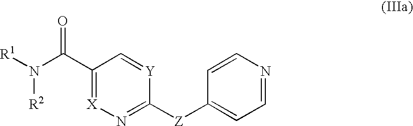

wherein all of the substituents have the meaning given in this specification.

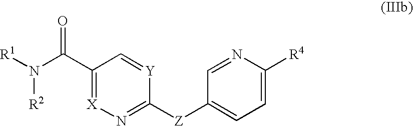

wherein the substituents have the meaning given in this specification.

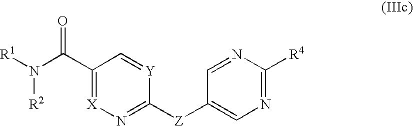

wherein the substituents have the meaning given in this specification.

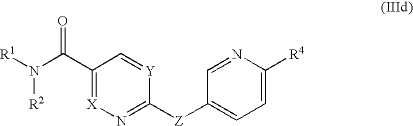

wherein R⁴ represents halogen; C₁-C₄alkyl, preferably methyl and the other substituents have the meaning given in this specification.

Where Q, V and W are all CR⁴, a preferred compound has a formula of (Ia):

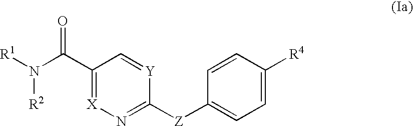

wherein R⁴ represents C₁-C₄alkyl; halogen, preferably chloro and the other substituents have the meaning given in this specification, including the preferences mentioned herein.

A particularly preferred class of compounds have the formulae (IV):

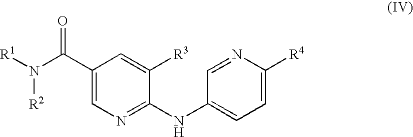

wherein the substituents have the meaning given in this specification, including the preferences mentioned herein.

In one class of compounds having the formula (IV), R¹ and R² form together with the Nitrogen atom to which they are attached very particularly preferably represent a heterocycle, as described herein. In particular, the heterocycle is unsubstituted, a single or twofold substituted.

In another class of compounds having the formula (IV), R³ is preferably halogen, e.g. chloro.

In a further class of compounds having the formula (IV), R⁴ is preferably alkyl, e.g. methyl.

In a further aspect, the invention provides process for the production of the compounds of formulae (I), (II), (III) and (IV) and their salts.

A first process, wherein Z represents NH or O, comprises the step of reacting a compound of formula (II)

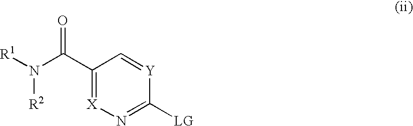

wherein R¹ and R² are as defined above, LG represents a leaving group such as Chlorine, Fluorine, methoxy, preferably chlorine, with a compound of formula (iii)

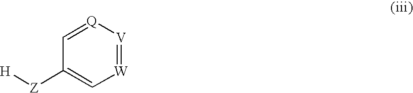

wherein Q, V, W is as defined above and Z represents NH or O, optionally in the presence of a reaction auxiliary,
and recovering the resulting compound in free base or acid addition salt form.

Such a process can be effected according to conventional methods, e.g. by aromatic nucleophilic substitution under acidic conditions as described in example 1. Optionally, the reaction is carried out under basic conditions in the presence or absence of a transition metal catalyst, e.g. by using for example potassium tert.-butoxide as base and palladium(II) acetate/BINAP catalyst as described in example 2.

Alternatively compounds of the invention, e.g. of formula (I), can be prepared by coupling an amine of formula (iv)

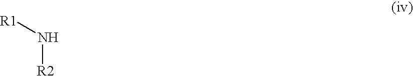

wherein R¹ and R² are defined as above and a carboxylic acid of formula (v)

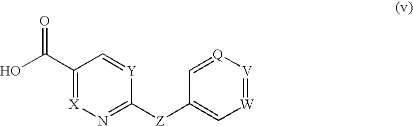

Q, V, W, Y, X and Z are defined as above.

Such a process can be performed by transforming acid (v) into an acyl halide (e.g. by thionyl chloride) which is then reacted with the desired amine (iv) to give (I) as outlined in example 4. Alternatively, acid (v) can be activated by a peptide coupling agent (e.g. HATU) and then converted to (I) by addition of an amine (iv) as shown in example 5.

Starting materials of formula (ii), (iii), (iv) and (v) are known or obtainable by known methods.

A further process for making compounds of the present invention, wherein Z represents CH₂, comprises the step of reacting a compound of formula (ii)

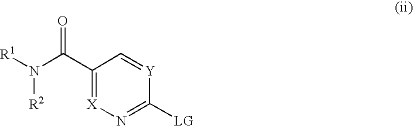

wherein R¹ and R² are as defined above, LG represents a leaving group such as Chlorine, Fluorine, methoxy, preferably chlorine, with a compound of formula (vi)

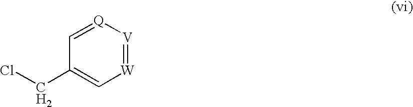

wherein Q, V, W is as defined above, optionally in the presence of a reaction auxiliary, such as a Zn/Ni(II) catalyst, e.g. Zn/NiCl₂(bisphosphine),
and recovering the resulting compound in free base or acid addition salt form.

Starting materials of formula (II) and (iv) are known or obtainable by known methods.

Compounds of the invention, e.g. of formula (II), are e.g. available by a process comprising the step of reacting a compound of formula (vii)

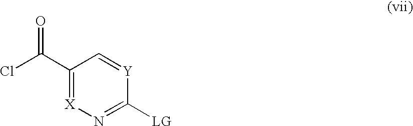

wherein X, Y and LG are as defined above with a compound of formula (iv)

R¹R²NH (iv)

wherein R¹ and R² are as defined above.

The starting materials of formula (vii) and (iv) are known or obtainable according to known methods. Instead of chlorine derivative (vi) the free acid in connection with an activating agent may be used. Such amid-formation reactions are known to the skilled person.

The following considerations apply to the individual reaction steps described above:

a) One or more functional groups, for example carboxy, hydroxy, amino, or mercapto, may need to be protected in the starting materials by protecting groups. The protecting groups employed may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter. The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosäuren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide und Derivate” (Chemistry of carbohydrates: monosaccharides and derivatives), Georg Thieme Verlag, Stuttgart 1974.

b) Acid addition salts may be produced from the free bases in known manner, and vice-versa. Compounds of formulae (I), (II), (III) and (IV) in optically pure form can be obtained from the corresponding racemates according to well-known procedures, e.g. HPLC with chiral matrix. Alternatively, optically pure starting materials can be used.

c) Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

d) Suitable diluents for carrying out the above-described are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile propionitrile or butyronitrile; amides, such as N,N-dimethylformamide, N,N-dimethylacetamide, N-methyl-formanilide, N-methyl-pyrrolidone or hexamethylphosphoric triamide; esters, such as methyl acetate or ethyl acetate, sulphoxides, such as dimethyl sulphoxide, alcohols, such as methanol, ethanol, n- or i-propanol, ethylene glycol monomethyl ether, ethylene glycol monoethyl ether, diethylene glycol monomethyl ether, diethylene glycol monoethyl ether. Further, mixtures of diluents may be employed. Depending on the starting materials, reaction conditions and auxiliaries, water or diluents constraining water may be suitable. It is also possible to use one a starting material as diluent simultaneously.

e) Reaction temperatures can be varied within a relatively wide range. In general, the processes are carried out at temperatures between 0° C. and 150° C., preferably between 10° C. and 120° C. Deprotonation reactions can be varied within a relatively wide range. In general, the processes are carried out at temperatures between −150° C. and +50° C., preferably between −75° C. and 0° C.

f) The reactions are generally carried out under atmospheric pressure. However, it is also possible to carry out the processes according to the invention under elevated or reduced pressure—in general between 0.1 bar and 10 bar.

g) Starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred at the required temperature for a number of hours.

h) Work-up is carried out by customary methods (cf. the Preparation Examples).

i) A compound of formulae (I), (II), (III) and (IV) obtained according to the above described processes can be converted into another compound formulae (I), (II), (III) and (IV) according to conventional methods.

Compounds of formulae (I), (II), (III) and (IV) and their pharmaceutically acceptable acid addition salts, hereinafter referred to as agents of the invention, exhibit valuable pharmacological properties and are therefore useful as pharmaceuticals.

In particular, the agents of the invention exhibit a marked and selective modulating, especially antagonistic, action at human metabotropic glutamate receptors (mGluRs). This can be determined in vitro for example at recombinant human metabotropic glutamate receptors, especially PLC-coupled subtypes thereof such as mGluR5, using different procedures like, for example, measurement of the inhibition of the agonist induced elevation of intracellular Ca²⁺ concentration in accordance with L. P. Daggett et al., Neuropharm. Vol. 34, pages 871-886 (1995), P. J. Flor et al., J. Neurochem. Vol. 67, pages 58-63 (1996) or by determination to what extent the agonist induced elevation of the inositol phosphate turnover is inhibited as described by T. Knoepfel et al., Eur. J. Pharmacol. Vol. 288, pages 389-392 (1994), L. P. Daggett et al., Neuropharm. Vol. 67, pages 58-63 (1996) and references cited therein. Isolation and expression of human mGluR subtypes are described in U.S. Pat. No. 5,521,297. Selected agents of the invention show IC50 values for the inhibition of the agonist (e.g. glutamate or quisqualate) induced elevation of intracellular Ca2+ concentration or the agonist (e.g. glutamate or quisqualate) induced inositol phosphate turnover, measured in recombinant cells expressing hmGluR5a of about 1 nM to about 50 μM.

The agents of the invention are therefore useful in the treatment of disorders associated with irregularities of the glutamatergic signal transmission, and of nervous system disorders mediated full or in part by mGluR5.

The agents of the invention are therefore useful in the prevention, treatment or delay of progression of disorders associated with irregularities of the glutamatergic signal transmission, of the gastro-intestinal and urinary tract and of nervous system disorders mediated full or in part by mGluR5.

Disorders associated with irregularities of the glutamatergic signal transmission are for example epileptogenesis including neuronal protection after status epilepticus, cerebral ischemias, especially acute ischemias, ischemic diseases of the eye, muscle spasms such as local or general spasticity, skin disorders, obesity disorders and, in particular, convulsions or pain.

Disorders of the gastro-intestinal tract include Gastro-Esophageal Reflux Disease (GERD), Functional Gastro-intestinal Disorders and Post-operative Ileus.

Functional Gastro-intestinal Disorders (FGIDs) are defined as chronic or recurrent conditions associated with abdominal symptoms without organic cause using conventional diagnostic measures. A cardinal symptom present in many FGIDs is visceral pain and/or discomfort. FGIDs include functional dyspepsia (FD), functional heartburn (a subset of GERD), irritable bowel syndrome (IBS), functional bloating, functional diarrhea, chronic constipation, functional disturbancies of the biliary tract as well as other conditions according to Gut 1999; Vol. 45 Suppl. II.

Post-operative Ileus is defined as failure of aboral passage of intestinal contents due to transient impairment of GI motility following abdominal surgery.

Disorders of the Urinary Tract comprise conditions associated with functional disturbancies and/or discomfort/pain of the urinary tract. Examples of disorders of the urinary tract include but are not limited to incontinence, benign prostatic hyperplasia, prostatitis, detrusor hyperreflexia, outlet obstruction, urinary frequency, nocturia, urinary urgency, overactive bladder (OAB), pelvic hypersensitivity, urge incontinence, urethritis, prostatodynia, cystitis, idiopathic bladder hypersensitivity and the like. OAB is a syndrome characterized by urgency, with or without urinary incontinence, and usually with increased voiding frequency and nocturia.

Nervous system disorders mediated full or in part by mGluR5 are for example acute, traumatic and chronic degenerative processes of the nervous system, such as Parkinson's disease, senile dementia, Alzheimer's disease, Huntington's chorea, amyotrophic lateral sclerosis, multiple sclerosis and fragile X syndrome, substance-related disorders, psychiatric diseases such as schizophrenia, affective and anxiety disorders, attention deficit disorders and cognitive dysfunction associated with these and other CNS disorders. Substance-related disorders include substance abuse, substance dependence and substance withdrawal disorders. Anxiety disorders includes panic disorder, social and specific phobias, anxiety, obsessive compulsive disorder (OCD), post traumatic stress disorder (PTSD) and generalized anxiety disorder (GAD). Affective disorders include depressive (major depression, dysthymia, depressive disorders NOS) and bipolar disorders (bipolar I and II disorders). Cognitive dysfunction associated with these and other CNS disorders include deficits and abnormalities in attention and vigilance, executive functions and memory (for instance working memory and episodic memory). Other disorders which are mediated fully or in part are pain and itch.

A further disorder is migraine.

The usefulness of the agents of the invention in the treatment of the above-mentioned disorders can be confirmed in a range of standard tests including those indicated below:

Activity of the agents of the invention in anxiety can be demonstrated in standard models such as the stress-induced hyperthermia in mice [cf. A. Lecci et al., Psychopharmacol. 101, 255-261]. At doses of about 0.1 to about 30 mg/kg p.o., selected agents of the invention reverse the stress-induced hyperthermia.

At doses of about 4 to about 50 mg/kg p.o., selected agents of the invention show reversal of Freund complete adjuvant (FCA) induced hyperalgesia [cf. J. Donnerer et al., Neuroscience 49, 693-698 (1992) and C. J. Woolf, Neuroscience 62, 327-331 (1994)].

Activity of the agents of the invention in GERD can be demonstrated in standard models such as the gastric distension-induced transient lower esophageal sphincter relaxations (TLESRs) in dogs. At doses of about 0.03 to about 10 mg/kg p.o., selected agents of the invention reduce the occurrence of TLESRs.

Activity of the agents of the invention in functional dyspepsia can be demonstrated a model of fasted gastric tone and gastric accommodation to meal in dogs. At doses of about 0.03 to about 10 mg/kg p.o., selected agents of the invention increase the gastric volume in fasting conditions indicative of a reduced gastric tone.

Activity of the agents of the invention in visceral hyperalgesia can be demonstrated in standard rat models according to modified methods by Tarrerias, A. et al., Pain-(2002) 100: 91-97, Schwetz, I. et al., Am. J. Physiol. (2005) 286: G683-G691, of La, J. et al., World J. Gastroenterol. (2003) 9: 2791-2795. At doses of about 0.03 to about 30 mg/kg p.o., selected agents of the invention reduce the exaggerated abdominal striated muscle contractions, indicative of a visceral antinociceptive activity.

Activity of the agents of the invention in visceral sensation/pain of the urinary bladder can be demonstrated in a standard mouse model according to a modified method by Ness T J and Elhefni H. J Urol. (2004) 171:1704-8. At doses of about 0.3 to about 30 mg/kg p.o., selected agents of the invention reduce the EMG (visceromotor) response, indicative of a visceral antinociceptive and/or hyposensitivity.

Activity of the agents of the invention in overactive bladder and urge incontinence can be demonstrated in standard cystometry models in rats according to modified method by Tagaki-Matzumoto et al J. Pharmacol. Sci. (2004) 95: 458-465. At doses of about 0.03 to about 10 mg/kg p.o., selected agents of the invention increased threshold volumes eliciting bladder contractions indicative of therapeutic potential in conditions with bladder dysfunctions.

For all the above mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.05 to about 100 mg/kg animal body weight. In larger mammals, for example humans, an indicated daily dosage is in the range from about 5 to 1500 mg, preferably about 10 to about 1000 mg of the compound conveniently administered in divided doses up to 4 times a day or in sustained release form.

In accordance with the foregoing, the present invention also provides in a further aspect an agent of the invention for use as a pharmaceutical, e.g. in the treatment of disorders associated with irregularities of the glutamatergic signal transmission, and of nervous system disorders mediated full or in part by mGluR5.

The invention also provides the use of an agent of the invention, in the treatment of disorders associated with irregularities of the glutamatergic signal transmission, and of nervous system disorders mediated full or in part by mGluR5.

In a further aspect, the invention provides the use of compounds of formula (I) as modulators of metabotropic Glutamate Receptors, Subtype 5 (“mGluR5-Modulators”).

Furthermore the invention provides the use of an agent of the invention for the manufacture of a pharmaceutical composition designed for the treatment of disorders associated with irregularities of the glutamatergic signal transmission, and of nervous system disorders mediated full or in part by mGluR5.

In a further aspect the invention relates to a method of treating disorders mediated full or in part by mGluR5, which method comprises administering to a warm-blooded organism in need of such treatment a therapeutically effective amount of an agent of the invention.

Moreover the invention relates to a pharmaceutical composition comprising an agent of the invention in association with one or more pharmaceutical carrier or one or more pharmaceutically acceptable diluent.

The pharmaceutical compositions according to the invention are compositions for enteral, such as nasal, rectal or oral, or parenteral, such as intramuscular or intravenous, administration to warm-blooded animals (human beings and animals) that comprise an effective dose of the pharmacological active ingredient alone or together with a significant amount of a pharmaceutically acceptable carrier. The dose of the active ingredient depends on the species of warm-blooded animal, body weight, age and individual condition, individual pharmacokinetic data, the disease to be treated and the mode of administration.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Preferred are the compounds according to the examples.

Further, properly isotope-labeled agents of the invention exhibit valuable properties as histopathological labeling agents, imaging agents and/or biomarkers, hereinafter “markers”, for the selective labeling of the metabotropic glutamate receptor subtype 5 (mGlu5 receptor). More particularly the agents of the invention are useful as markers for labeling the central and peripheral mGlu5 receptors in vitro or in vivo. In particular, compounds of the invention which are properly isotopically labeled are useful as PET markers. Such PET markers are labeled with one or more atoms selected from the group consisting of ¹¹C, ¹³N, ¹⁵O, ¹⁸F.

The agents of the invention are therefore useful, for instance, for determining the levels of receptor occupancy of a drug acting at the mGlu5 receptor, or diagnostic purposes for diseases resulting from an imbalance or dysfunction of mGlu5 receptors, and for monitoring the effectiveness of pharmacotherapies of such diseases.

In accordance with the above, the present invention provides an agent of the invention for use as a marker for neuroimaging.

In a further aspect, the present invention provides a composition for labeling brain and peripheral nervous system structures involving mGlu5 receptors in vivo and in vitro comprising an agent of the invention.

In still a further aspect, the present invention provides a method for labeling brain and peripheral nervous system structures involving mGlu5 receptors in vitro or in vivo, which comprises contacting brain tissue with an agent of the invention.

The method of the invention may comprise a further step aimed at determining whether the agent of the invention labeled the target structure. Said further step may be effected by observing the target structure using positron emission tomography (PET) or single photon emission computed tomography (SPECT), or any device allowing detection of radioactive radiations.

The following non-limiting Examples illustrate the invention. A list of Abbreviations used is given below.

ACOH acetic acid

BOC tert-butoxycarbonyl

n-BuLi n-butyl lithium

DMF N,N′-dimethylformamide

EDC 1-ethyl-3-[3-(dimethylamino)propyl]-carbodiimide hydrochloride

HOBt hydroxybenzotriazole

AcN acetonitrile

BINAP (2,2′-bis(diphenylphosphino)-1,1′-binaphthyl

DAST (Diethylamino)sulfur trifluoride

DCE 1,2-dichloroethane

DCM dichloromethane

DIPEA N,N-diisopropylethylamine

DMA N,N-dimethylacetamide

DMAP 4-N,N-dimethylaminopyridin

DME 1,2-dimethoxyethane

DMSO dimethylsulfoxide

EtOAc ethylacetate

ESI electrospray ionization

h hours

HCl hydrochloric acid

HATU N-[(dimethylamino)-1H-1,2,3-triazolo[4,5-b]pyridin-1-ylmethylene]-N-methyl-methanaminium hexafluorophosphate N³-oxide

HMPA hexamethylphosphoramide

HPLC high pressure liquid chromatography

min minutes

Mp melting point

MS mass spectroscopy

MTBE methyl-tert.-butylether

R_{f} retention factor (Thin Layer Chromatography)

rt room temperature

t_{R} retention time

TFA trifluoroacetic acid

THF tetrahydrofuran

HPLC Specificity

System 1: System 1: Performed on a Waters system equipped with a CTC Analytics HTS PAL autosampler, 515 pumps, and a 996 DAD detector operating at 210 nm. Column: CC70/3 Nucleosil 100-3 C₁₈ (3 p, 70×3 mm, Macherey-Nagel, order # 721791.30), temperature: 45° C., flow: 1.2 mL min⁻¹. Eluents: A: Water+0.2% H₃PO₄ (85%, (Merck 100552)+2% Me₄NOH, (10%, Merck 108123), B: Acetonitrile+20% water+0.1% H₃PO₄ (85%)+1% Me₄NOH (10%). Gradient: 0% B to 95% B within 6.6 min., then 95% B 4.4 min. System 2: Gilson 331 pumps coupled to a Gilson UV/NIS 152 detector and a Finnigan AQA spectrometer (ESI), a 50 μL loop injection valve and a Waters XTerra MS C18 3.5 μm 4.6×50 mm column running a gradient from 5% to 90% acetonitrile containing 0.05% TFA. System 3: Agilent 1100 Series, LC-MSD and a Agilent Zorbax SB-C18 3×30 mm 1.8 μm Column running a gradient Water+0.05% TFA/Acetonitrile+0.05% TFA from 100/0 to 0/100 over 3.25′-0/100 over 0.75′-0/100 to 90/10 over 0.25′ with a flux of 0.7 ml/min, 35° C.

System 4: Agilent 1100 Series, LC-MSD and a Agilent Zorbax SB-C18 3×30 mm 1.8 μm Column running a gradient Water+0.05% TFA/Acetonitrile+0.05% TFA from 90/10 to 0/100 over 3.25′-0/100 over 0.75′-0/100 to 70/30 over 0.25′ with a flux of 0.7 ml/min, 35° C.

System 5: Agilent 1100 Series, LC-MSD and a Agilent Zorbax SB-C18 3×30 mm 1.8 μm Column running a gradient Water+0.05% TFA/Acetonitrile+0.05% TFA from 70/30 to 0/100 over 3.25′-0/100 over 0.75′-0/100 to 60/40 over 0.25′ with a flux of 0.7 ml/min, 35° C.

System 6: Agilent 1100 Series, LC-MSD and a Agilent Zorbax SB-C18 3×30 mm 1.8 μm Column running a gradient Water+0.05% TFA/Acetonitrile+0.05% TFA from 30/70 to 0/100 over 3.25′-0/100 over 0.75′-0/100 to 90/10 over 0.25′ with a flux of 0.7 ml/min, 35° C.

### EXAMPLE 1.1

### 6-(4-Chloro-phenylamino)-N,N-diethyl-nicotinamide hydrochloride

6-Chloro-N,N-diethyl-nicotinamide (100 mg, 0.47 mmol) and 4-chloroaniline (184 mg, 1.41 mmol) are suspended in a mixture of glacial acetic acid (0.6 mL) and water (1.4 mL). The reaction mixture is heated in a sealed 3 mL-vial to 100° C. over night. After reaching room temperature the reaction mixture is poured onto MTBE (30 mL) and extracted with 2M HCl (3×5 mL). The combined acidic extracts are made alkaline by addition of 2M NaOH (10 mL) extracted with MTBE (3×15 mL). The combined organic extracts are dried (Na₂SO₄) and evaporated to dryness to. The residue is purified by flash-chromatography. To the combined product containing fractions is added 4M HCl in dioxane (0.25 mL) followed by evaporation. The residue is triturated with ether, filtered off, washed with cold ether and vacuum dried at 45° C. to give the title compound as colorless crystals (90 mg, 56%). TLC: R_{f}=0.16 (MTBE), HPLC: t_{R}=6.0 min, (system 1); ESI+MS: m/z=304.5 (MH⁺).

The starting material can be prepared as described hereafter:

6-Chloro-N,N-diethyl-nicotinamide

Under Ar, chloronicotinoyl chloride (4 g, 22 mmol) is suspended in DCM (40 mL). The reaction flask is placed in an ice bath and a solution of diethylamine (2.31 mL, 22 mmol) and triethylamine (3.90 mL, 27.8 mmol) in DCM (40 mL) is added within 45 min keeping the internal temperature below 5° C. The ice bath is removed and the reaction mixture is stirred for further 30 min. The solution is washed (1× water (40 mL), 1×μM Na₂CO₃ (40 mL), 1× water (40 mL)), dried over Na₂SO₄ and evaporated to dryness to afford a reddish orange oil (4.50 g, 95%) which can be used without further purification.

Following the same procedure, the following compounds can be prepared:

### EXAMPLE 1.2

### N,N-Diethyl-6-p-tolylamino-nicotinamide hydrochloride

Yellowish lyophilisate, TLC: R_{f}=0.22 (MTBE), HPLC: t_{R}=5.5 min, (system 1); ESI+MS: m/z=284.6 (MH⁺).

### EXAMPLE 1.3

### N,N-Diethyl-6-(4-methoxy-phenylamino)-nicotinamide hydrochloride

Light gray crystals, TLC: R_{f}=0.14 (MTBE), HPLC: t_{R}=4.6 min, (system 1); ESI+MS: m/z=300.6 (MH⁺).

### EXAMPLE 1.4

### 6-(4-Chloro-phenylamino)-N,N-bis-(2-methoxy-ethyl)-nicotinamide hydrochloride

Yellowish lyophilisate, TLC: R_{f}=0.10 (MTBE), HPLC: t_{R}=5.6 min, (system 1); ESI+MS: m/z=364.5 (MH⁺).

### EXAMPLE 1.5

### [6-(4-Chloro-3-fluoro-phenylamino)-pyridin-3-yl]-piperidin-1-yl-methanone

Colorless crystals, HPLC: t_{R}=6.6 min, (system 1); ESI+MS: m/z=334.5 (MH⁺).

### EXAMPLE 1.6

### [6-(4-Bromo-phenylamino)-pyridin-3-yl]-piperidin-1-yl-methanone

Colorless crystals, TLC: R_{f}=0.31 (MTBE-ETOAC 9:1), HPLC: t_{R}=6.3 min, (system 1); ESI+MS: m/z=360.6 (MH⁺).

### EXAMPLE 1.7

### 4-[5-(Piperidine-1-carbonyl)-pyridin-2-ylamino]-benzonitrile

Colorless crystals, TLC: R_{f}=0.14 (MTBE), HPLC: t_{R}=5.7 min, (system 1); ESI+MS: m/z=307.6 (MH⁺).

### EXAMPLE 1.8

### Piperidin-1-yl-[6-(4-trifluoromethoxy-phenylamino)-pyridin-3-yl]-methanone

Colorless crystals, TLC: R_{f}=0.29 (DCM-ETOAC 7:3), HPLC: t_{R}=6.6 min, (system 1); ESI+MS: m/z=366.7 (MH⁺).

### EXAMPLE 1.9

### [6-(4-Chloro-phenylamino)-pyridin-3-yl]-(2-methyl-piperidin-1-yl)-methanone hydrochloride

TLC: R_{f}=0.23 (MTBE), HPLC: t_{R}=6.5 min, (system 1); ESI+MS: m/z=330.5 (MH⁺).

### EXAMPLE 1.10

### (2-Methyl-piperidin-1-yl)-(6-p-tolylamino-pyridin-3-yl)-methanone

Beige crystals, TLC: R_{f}=0.24 (MTBE), HPLC: t_{R}=6.0 min, (system 1); ESI+MS: m/z=310.5 (MH⁺).

### EXAMPLE 1.11

### [6-(4-Methoxy-phenylamino)-pyridin-3-yl]-(2-methyl-piperidin-1-yl)-methanone hydrochloride

Purple crystals, TLC: R_{f}=0.27 (MTBE), HPLC: t_{R}=5.4 min, (system 1); ESI+MS: m/z=326.5 (MH⁺).

### EXAMPLE 1.12

### rac-[6-(4-Chloro-phenylamino)-pyridin-3-yl]-(3-methyl-piperidin-1-yl)-methanone

Colorless crystals, TLC: R_{f}=0.25 (MTBE), HPLC: t_{R}=6.6 min, (system 1); ESI+MS: m/z=330.5 (MH⁺).

Using either S-3-methylpiperidine or R-3-methylpiperidine as starting material the pure enantiomers could be prepared:

### EXAMPLE 1.12a

### [6-(4-Chloro-phenylamino)-pyridin-3-yl]-(S-3-methyl-piperidin-1-yl)-methanone

Colorless crystals, TLC: R_{f}, =0.22 (MTBE), HPLC: t_{R}=6.7 min, (system 1); ESI+MS: m/z=330.1 (MH⁺).

### EXAMPLE 1.12b

### [6-(4-Chloro-phenylamino)-pyridin-3-yl]-(R-3-methyl-piperidin-1-yl)-methanone

Beige crystals, HPLC: t_{R}=6.7 min, (system 1); ESI+MS: m/z=330.2 (MH⁺).

### EXAMPLE 1.13

### 3-Methyl-piperidin-1-yl)-(6-p-tolylamino-pyridin-3-yl)-methanone

Pink lyophilisate, HPLC: t_{R}=6.2 min, (system 1); ESI+MS: m/z=310.5 (MH⁺).

### EXAMPLE 1.14

### [6-(4-Methoxy-phenylamino)-pyridin-3-yl]-(3-methyl-piperidin-1-yl)-methanone hydrochloride

Brown crystals, HPLC: t_{R}=5.6 min, (system 1); ESI+MS: m/z=326.5 (MH⁺).

### EXAMPLE 1.15

### (3-Methyl-piperidin-1-yl)-(6-phenylamino-pyridin-3-yl)-methanone hydrochloride

Colorless crystals, TLC: R_{f}=0.26 (MTBE), HPLC: t_{R}=5.8 min, (system 1); ESI+MS: m/z=296.5 (MH⁺).

### EXAMPLE 1.16

### [6-(3-Chloro-phenylamino)-pyridin-3-yl]-(3-methyl-piperidin-1-yl)-methanone hydrochloride

TLC: R_{f}=0.27 (MTBE), HPLC: t_{R}=6.6 min, (system 1); ESI+MS: m/z=330.5 (MH⁺).

### EXAMPLE 1.17

### [6-(4-Chloro-phenylamino)-pyridin-3-yl]-morpholin-4-yl-methanone hydrochloride

Yellowish crystals, TLC: R_{f}=0.38 (MTBE-MeOH 9:1), HPLC: t_{R}=5.5 min, (system 1); ESI+MS: m/z=318.5 (MH⁺).

### EXAMPLE 1.18

### [6-(4-Methoxy-phenylamino)-pyridin-3-yl]-morpholin-4-yl-methanone hydrochloride

Greenish solid, TLC: R_{f}=0.35 (MTBE-MeOH 9:1), HPLC: t_{R}=4.0 min, (system 1); ESI+MS: m/z=314.5 (MH⁺).

### EXAMPLE 1.19

### cis-[6-(4-Chloro-phenylamino)-pyridin-3-yl]-(2,6-dimethyl-morpholin-4-yl)-methanone hydrochloride

Colorless crystals, TLC: R_{f}=0.13 (MTBE), HPLC: t_{R}=6.1 min, (system 1); ESI+MS: m/z=346.5 (MH⁺).

### EXAMPLE 1.20

### (cis-2,6-Dimethyl-morpholin-4-yl)-(6-p-tolylamino-pyridin-3-yl)-methanone hydrochloride

Beige crystals, TLC: R_{f}=0.24 (MTBE), HPLC: t_{R}=5.4 min, (system 1); ESI+MS: m/z=326.6 (MH⁺).

### EXAMPLE 1.21

### (cis-Z 6-Dimethyl-morpholin-4-yl)-[6-(4-methoxy-phenylamino)-pyridin-3-yl]-methanone hydrochloride

Purple crystals, TLC: R_{f}=0.16 (MTBE), HPLC: t_{R}=4.9 min, (system 1); ESI+MS: m/z=342.5 (MH⁺).

### EXAMPLE 2.1

### [6-(5-Chloro-pyridin-2-ylamino)-pyridin-3-yl]-piperidin-1-yl-methanone

A solution of palladium(II) acetate (2 mg, 9 μmol) and BINAP (5.6 mg, 9 μmol) in dry and degassed toluene (1.5 mL) is stirred for 10 min under Ar. Then, the clear yellow solution obtained is added to a degassed suspension of (6-chloro-pyridin-3-yl)-piperidin-1-yl-methanone (100 mg, 0.45 mmol, prepared according to the general procedure stated in example 1.1), 2-amino-5-chloropyridin (70 mg, 0.53 mmol), and KOtBu (257 mg, 2.22 mmol) in dry toluene. The reaction mixture is stirred for 5 h in a sealed 5 mL-vial. After reaching room temperature the mixture is poured into MTBE (30 mL), washed (3×H₂O (20 mL)), dried over Na₂SO₄ and evaporated to give a turbid oil. Crystallization from Et₂O affords the title compound as beige crystals (87 mg, 62%), HPLC: t_{R}=4.8 min, (system 1); ESI+MS: m/z=317.6 (MH⁺).

Following the same procedure, the following compounds can be prepared:

### EXAMPLE 2.2

### Azepan-1-yl-[6-(pyridin-3-ylamino)-pyridin-3-yl]-methanone

Yellowish lyophilisate, TLC: R_{f}=0.28 (MTBE-MeOH 85:15), HPLC: t_{R}=4.2 min, (system 1); ESI+MS: m/z=297.2 (MH⁺).

### EXAMPLE 2.3

### [6-(3,4-Difluoro-phenylamino)-pyridin-3-yl]-piperidin-1-yl-methanone

Colorless crystals, HPLC: t_{R}=6.1 min, (system 1); ESI+MS: m/z=318.6 (MH⁺).

### EXAMPLE 2.4

### rac-(2-Aza-bicyclo[2.2.1]hept-2-yl)-[5-chloro-6-(4-chloro-phenylamino)-pyridin-3-yl]-methanone

Beige powder, HPLC: t_{R}=6.9 min, (system 1); ESI+MS: m/z=364.0 (MH⁺).

### EXAMPLE 2.5

### [5-Chloro-6-(4-chloro-phenylamino)-pyridin-3-yl]-thiomorpholin-4-yl-methanone

Beige powder, HPLC: t_{R}=6.6 min, (system 1); ESI+MS: m/z=370.0 (MH⁺).

### EXAMPLE 2.6

### rac-[5-Chloro-6-(6-methoxy-pyridin-3-ylamino)-pyridin-3-yl]-(3-methyl-piperidin-1-yl)-methanone

Beige lyophilisate, TLC: R_{f}=0.49 (MTBE), HPLC: t_{R}=6.2 min (system 1); ESI+MS: m/z=361.1 (MH⁺).

### EXAMPLE 2.7

### Azepan-1-yl-[5-chloro-6-(6-methoxy-pyridin-3-ylamino)-pyridin-3-yl]-methanone

Beige lyophilisate, TLC: R_{f}=0.32 (MTBE), HPLC: t_{R}=6.0 min (system 1); ESI+MS: m/z=361.1 (MH⁺).

### EXAMPLE 2.8

### [5-Chloro-6-(6-methoxy-pyridin-3-ylamino)-pyridin-3-yl]-piperidin-1-yl-methanone

Colorless lyophilisate, TLC: R_{f}=0.36 (MTBE), HPLC: t_{R}=5.9 min (system 1); ESI+MS: m/z=347.0 (MH⁺).

### EXAMPLE 2.9

### [5-Chloro-6-(6-ethoxy-pyridin-3-ylamino)-pyridin-3-yl]-piperidin-1-yl-methanone

Colorless lyophilisate, TLC: R_{f}=0.23 (EtOAc/hexanes 1:1), LC/MS: m/z=361 (MH⁺).

### EXAMPLE 2.10

### rac-[5-Chloro-6-(6-methyl-pyridin-3-ylamino)-pyridin-3-yl]-(3-methyl-piperidin-1-yl)-methanone

Beige crystals, HPLC: t_{R}=4.7 min (system 1); ESI+MS: m/z=345.1 (MH⁺).

Using either S-3-methylpiperidine or R-3-methylpiperidine as starting material the pure enantiomers could be prepared:

### EXAMPLE 2.10a

### [5-Chloro-6-(6-methyl-pyridin-3-ylamino)-pyridin-3-yl]-(S-3-methyl-piperidin-1-yl)-methanone

Brown gum, HPLC: t_{R}=4.7 min (system 1); ESI+MS. m/z=345.1 (MH⁺)

### EXAMPLE 2.10b

### [5-Chloro-6-(6-methyl-pyridin-3-ylamino)-pyridin-3-yl]-(R-3-methyl-piperidin-1-yl)-methanone

Brown gum, HPLC: t_{R}=4.5 min (system 1); ESI+MS. m/z=345.1 (MH⁺)

### EXAMPLE 2.11

### [5-Chloro-6-(6-methyl-pyridin-3-ylamino)-pyridin-3-yl]-piperidin-1-yl-methanone

Colorless crystals, HPLC: t_{R}=4.3 min (system 1); ESI+MS: m/z=331.1 (MH⁺).

### EXAMPLE 2.12

### Azepan-1-yl-[5-chloro-6-(6-methyl-pyridin-3-ylamino)-pyridin-3-yl]-methanone

Colorless crystals, HPLC: t_{R}=4.3 min (system 1); ESI+MS: m/z=345.1 (MH⁺).

### EXAMPLE 2.13

### rac-(2-Aza-bicyclo[2.2.1]hept-2-yl)-[5-chloro-6-(6-methyl-pyridin-3-ylamino)-pyridin-3-yl]-methanone

Beige powder, HPLC: t_{R}=4.1 min (system 1); ESI+MS: m/z=343.1 (MH⁺).

### EXAMPLE 2.14

### [5-Chloro-6-(6-methyl-pyridin-3-ylamino)-pyridin-3-yl]-thiazolidin-3-yl-methanone

Beige powder, HPLC: t_{R}=4.1 min (system 1); ESI+MS: m/z=335.0 (MH⁺).

### EXAMPLE 2.15

### [5-Chloro-6-(6-methyl-pyridin-3-ylamino)-pyridin-3-yl]-thiomorpholin-4-yl-methanone

Beige powder, HPLC: t_{R}=3.9 min (system 1); ESI+MS: m/z=349.0 (MH⁺).

### EXAMPLE 2.16

### [5-Chloro-6-(2-methyl-pyrimidin-5-ylamino)-pyridin-3-yl]-(3-methyl-piperidin-1-yl)-methanone

Colorless crystals, HPLC: t_{R}=5.6 min (system 1); ESI+MS: m/z=346.1 (MH⁺).

### EXAMPLE 2.17

### [5-Chloro-6-(2-methyl-pyrimidin-5-ylamino)-pyridin-3-yl]-piperidin-1-yl-methanone

Colorless crystals, HPLC: t_{R}=5.1 min (system 1); ESI+MS: m/z=332.1 (MH⁺).

### EXAMPLE 2.18

### Azepan-1-yl-[5-chloro-6-(2-methyl-pyrimidin-5-ylamino)-pyridin-3-yl]-methanone

Colorless crystals, HPLC: t_{R}=5.5 min (system 1); ESI+MS: m/z=346.1 (MH⁺).

### EXAMPLE 2.19

### [5-Chloro-6-(6-methyl-pyridin-3-ylamino)-pyridin-3-yl]-(3-ethyl-piperidin-1-yl)-methanone

A mixture of (5,6-dichloro-pyridin-3-yl)-(3-ethyl-piperidin-1-yl)-methanone (300 mg, 1.04 mmol), 3-amino-6-methylpyridin (171 mg, 1.57 mmol), Pd(OAc)₂ (7 mg, 0.03 mmol), rac-BINAP (20 mg, 0.03 mmol) and potassium carbonate (723 mg, 5.2 mmol) in degassed toluene (10 mL) was stirred, under argon, at 80° C. for 3 hours. EtOAc was added and the organic phase was washed with water, dried over sodium sulfate and concentrated in vacuo to give a crude beige powder. The crude material was sonicated in pentane/Et₂O and then filtered. After high-vacuum drying, [5-chloro-6-(6-methyl-pyridin-3-ylamino)-pyridin-3-yl]-(3-ethyl-piperidin-1-yl)-methanone (100 mg, 27%) was obtained as a beige powder. (ES-MS: m/z 359.3/361.3 [M+H]⁺, t_{R} 3.52 min (system 2)).

The starting material was prepared as described hereafter:

i) 3-ethyl piperidine

3-Ethyl pyridin (5.0 g, 46.7 mmol) was hydrogenated in AcOH (100 mL) over PtO₂ (500 mg) under 4 bar for 4 hours. The mixture was filtered through a pad of celite and washed with AcOH. The solvent was removed in vacuo and the residue was dissolved into water. The solution was basified by addition with 40% NaOH solution. The aqueous phase was extracted with Et₂O. The organic phases were combined, dried over sodium sulfate and concentrated in vacuo to afford 3-ethyl piperidine (4.4 g, 83%) as a clear yellow oil.

ii) (5,6-Dichloro-pyridin-3-yl)-(3-ethyl-piperidin-1-yl)-methanone

A mixture of 5,6 dichloronicotinic acid (1 g, 5.2 mmol) in SOCl₂ (6 mL) was stirred at 70° C. for 4 hours. The solvent was removed in vacuo to give a beige oil (1.05 g) corresponding to the acid chloride. This oil was solubilised in DCM (15 mL) and at 0° C. triethylamine (1.1 mL, 7.84 mmol) was added. Then, a solution of 3-ethyl piperidine (657 mg, 5.75 mmol) in DCM (5 mL) was added carefully drop-wise. At the end of the addition, the mixture was stirred at RT for 30 min. Water was added and the aqueous phase was extracted with DCM. The organic phases were combined, dried over sodium sulfate and concentrated in vacuo to (5,6-dichloro-pyridin-3-yl)-(3-ethyl-piperidin-1-yl)-methanone (1.2 g, 80%) as a yellow oil. (ES-MS: m/z 328.2/330.2 [M+CH₃CN+H]⁺, t_{R} 5.48 min (system 2)).

### EXAMPLE 2.20

### [5-Chloro-6-(6-methoxy-pyridin-3-ylamino)-pyridin-3-yl]-(3-ethyl-piperidin-1-yl)-methanone

A mixture of (5,6-dichloro-pyridin-3-yl)-(3-ethyl-piperidin-1-yl)-methanone (300 mg, 1.04 mmol), 5-amino-2-methoxy pyridin (201 mg, 1.57 mmol), Pd(OAc)₂ (7 mg, 0.03 mmol), rac-BINAP (20 mg, 0.03 mmol) and potassium carbonate (723 mg, 5.2 mmol) in degassed toluene (10 mL) was stirred, under argon, at 80° C. for 3 hours. EtOAc was added and the organic phase was washed with water, dried over sodium sulfate and concentrated in vacuo to give a crude beige powder. The crude material was purified by flash chromatography using EtOAc/Hexanes as eluent to afford [5-chloro-6-(6-methoxy-pyridin-3-ylamino)-pyridin-3-yl]-(3-ethyl-piperidin-1-yl)methanone (60 mg, 15%) as a beige powder. (ES-MS: m/z 375.3/375.5 [M+H]⁺, t_{R} 5.21 min (system 2))

### EXAMPLE 2.21

### [5-Chloro-6-(4-chloro-phenylamino)-pyridin-3-yl]-(3-ethyl-piperidin-1-yl)-methanone

A mixture of (5,6-dichloro-pyridin-3-yl)-(3-ethyl-piperidin-1-yl)-methanone (300 mg, 1.04 mmol), 4-chloro aniline (206 mg, 1.57 mmol), Pd(OAc)₂ (7 mg, 0.03 mmol), rac-BINAP (20 mg, 0.03 mmol) and potassium carbonate (723 mg, 5.2 mmol) in degassed toluene (10 mL) was stirred, under argon, at 80° C. for 3 hours. EtOAc was added and the organic phase was washed with water, dried over sodium sulfate and concentrated in vacuo to give a crude beige powder. The crude material was purified by flash chromatography using EtOAc/Hexanes as eluent to afford [5-chloro-6-(4-chloro-phenylamino)-pyridin-3-yl]-(3-ethyl-piperidin-1-yl)-methanone (150 mg, 38%) as a beige powder. (ES-MS: m/z 378.2/380.3 [M+H]⁺, t_{R} 6.50 min (system 2))

### EXAMPLE 2.22

### [5-Chloro-6(6-methyl-pyridin-3-ylamino)-pyridin-3-yl]-(3-propyl-piperidin-1-yl)-methanone

A mixture of (5,6-dichloro-pyridin-3-yl)-(3-propyl-piperidin-1-yl)-methanone (440 mg, 1.26 mmol), 3-amino-6-methylpyridin (210 mg, 1.88 mmol), Pd(OAc)₂ (8.6 mg, 0.03 mmol), rac-BINAP (24 mg, 0.03 mmol) and potassium carbonate (879 mg, 5.0 mmol) in degassed toluene (10 mL) was stirred, under argon, at 80° C. for 3 hours. EtOAc was added and the organic phase was washed with water, dried over sodium sulfate and concentrated in vacuo to give a crude beige powder. The crude material was purified by flash chromatography using EtOAc/Hexanes as eluent to afford [5-chloro-6-(6-methyl-pyridin-3-ylamino)-pyridin-3-yl]-(3-propyl-piperidin-1-yl)-methanone (110 mg, 23%) as a beige powder. (ES-MS: m/z 373.3/375.3 [M+H]⁺, t_{R} 4.52 min (system 2))

The starting material was prepared as described hereafter:

i) 3-propyl pyridin

At 0° C., to a mixture of diisopropylamine (3.53 mL, 24.7 mmol) in THF (35 mL), BuLi (1.6 M in hexanes, 15.4 mL, 25 mmol) was added drop-wise. After 30 min, HMPA (15.7 g, 24.7 mmol) was added and the mixture was kept at 0° C. for 15 min. Then a solution of 3-methyl pyridin (2.3 g, 24.7 mmol) in THF (10 mL) was added drop-wise. After 30 min, Etl (3.45 g, 24.7 mmol) in THF (10 mL) was added drop-wise and the mixture was then stirred at RT for 1 hour. The mixture was poured into 10% HCl. The aqueous phase was extracted with Et₂O. The organic phase was washed with water, dried over sodium sulfate and concentrated in vacuo to afford a yellow oil (300 mg, 10%) which will be used without further purification.

ii) 3-propyl piperidine

3-propyl pyridin (300 mg, 2.48 mmol) was hydrogenated in AcOH (20 mL) over PtO₂ (50 mg) under 4 bar for 16 hours. The mixture was filtered through a pad of celite and washed with AcOH. The solvent was removed in vacuo and the residue was dissolved into water. The solution was basified by addition with 40% NaOH solution. The aqueous phase was extracted with Et₂O. The organic phases were combined, dried over sodium sulfate and concentrated in vacuo to afford 3-propyl piperidine (300 mg, 95%) as a clear yellow oil.

iii) (5,6-Dichloro-pyridin-3-yl)-(3-propyl-piperidin-1-yl)-methanone

5,6-Dichloro-nicotinoyl chloride (550 mg, 2.61 mmol) was solubilised in DCM (15 mL) and at 0° C. triethylamine (0.54 mL, 3.95 mmol) was added. Then, a solution of 3-propyl piperidine (369 mg, 2.87 mmol) in DCM (5 mL) was added carefully drop-wise. At the end of the addition, the mixture was stirred at RT for 30 min. Water was added and the aqueous phase was extracted with DCM. The organic phases were combined, dried over sodium sulfate and concentrated in vacuo to afford a beige-brown oil. This oil was sonicated in pentane to afford (5,6-dichloro-pyridin-3-yl)-(3-propyl-piperidin-1-yl)-methanone (440 mg, 48%) as a beige-brown solid.

### EXAMPLE 2.23

### [5-Chloro-6-(6-trifluoromethyl-pyridin-3-ylamino)-pyridin-3-yl]-((R)-2-ethyl-piperidin-1-yl)-methanone

LC/MS: m/z=413 (MH⁺); TLC: R_{f}=0.40 (DCM/MeOH 95:5).

### EXAMPLE 2.24

### [5-Chloro-6-(6-methoxy-pyridin-3-ylamino)-pyridin-3-yl]-((R)-2-ethyl-piperidin-1-yl)-methanone

LC/MS: m/z=375 (MH⁺); TLC: R_{f}=0.40 (DCM/MeOH 95:5).

### EXAMPLE 3.1

### rac-[5-Chloro-6-(6-methyl-pyridin-3-yloxy)-pyridin-3-yl]-(3-methyl-piperidin-1-yl)-methanone

To a solution of rac-(5,6-dichloropyridin-3-yl)-(3-methyl-piperidin-1-yl)-methanone (50 mg, 0.18 mmol, prepared according to the procedure stated in example 1.1) and 4-chlorophenol (23.5 mg, 0.18 mmol) in dry DMA (1 mL) is added finely ground anhydrous K₂CO₃ (50.6 mg, 0.36 mmol). The suspension is microwave heated to 140° C. in a sealed 5 mL-vial for 45 min with stirring. Then, the reaction mixture is diluted with ethyl acetate (10 mL) and washed with brine (10 mL). The organic layer is dried (Na₂SO₄) and evaporated to dryness to give a brown oil. Purification by preparative HPLC afforded the title compound as colorless syrup (40 mg, 60%), HPLC: t_{R}=7.1 min (system 1); ESI+MS: m/z=365.0 (MH⁺).

Following the same procedure, the following compounds can be prepared:

### EXAMPLE 3.2

### [5-Chloro-6-(6-methyl-pyridin-3-yloxy)-pyridin-3-yl]-piperidin-1-yl-methanone

Colorless syrup, HPLC: t_{R}=6.8 min (system 1); ESI+MS: m/z=351.0 (MH⁺).

### EXAMPLE 3.3

### Azepan-1-yl-[5-chloro-6-(6-methyl-pyridin-3-yloxy)-pyridin-3-yl]-methanone

Colorless syrup, HPLC: t_{R}=7.0 min (system 1); ESI+MS: m/z=365.0 (MH⁺).

### EXAMPLE 4.1

### [6-(6-Methyl-pyridin-3-ylamino)-pyridin-3-yl]-piperidin-1-yl-methanone

To 6-(6-methyl-pyridin-3-ylamino)-nicotinic acid (210 mg, 0.92 mmol) is added thionyl chloride (2 mL). The colorless suspension is refluxed under argon for 20 min. After cooling the excess thionyl chloride is stripped off. The residue is redissolved in DCM (6 mL) and a solution of piperidine (0.11 mL, 1.10 mmol) and triethylamine (1.28 mL, 9.16 mmol) in DCM (2 mL) is quickly added. The yellow slightly turbid solution is stirred for 20 min at room temperature. Then, MTBE (60 mL) is added and the solution is extracted twice with water and brine. The organic layer is dried over Na₂SO₄ and evaporated to give a yellow foam. Flash chromatography (20 g silica gel, MeOH-MTBE gradient 2%->15% MeOH, flow 20 mL min⁻¹) followed by crystallization from ether affords the title compound as colorless crystals (573 mg, 63%), TLC: R_{f}=0.18 (MTBE-MeOH 9:1), HPLC: t_{R}=3.8 min (system 1); ESI+MS: m/z=297.5 (MH⁺).

The starting material can be prepared as described hereafter:

i) Methyl 6-(6-Methyl-pyridin-3-ylamino)-nicotinate

To 5-amino-2-methylpyridin (2.22 g, 20.56 mmol) and finely ground anhydrous K₂CO₃ (11.9 g, 85.2 mmol) is added dry toluene (30 mL) under argon. Then, a solution of palladium(II) acetate (79 mg, 0.34 mmol) and BINAP (218 mg, 0.34 mmol) in dry toluene (10 mL) is added. The reaction mixture is placed in an oil bath (70° C.) and a solution of methyl 6-chloronicotinate (3.0 g, 17.1 mmol) in dry toluene (20 mL) is added dropwise within 30 min. After 1.5 h the oilbath is removed and the reaction flask is placed in an ice bath. After stirring for 15 min the product is filtered off. The filter cake is triturated three times with THF/MeOH 1:1 (100 mL). The combined extracts are evaporated to dryness to give a brown powder. Flash chromatography (gradient MTBE-MeOH 100:0-MTBE-MeOH 85:15) followed by crystallization from ether gives the product as light pink crystals (1.86 g, 45%).

ii) 6-(6-Methyl-pyridin-3-ylamino)-nicotinic acid

To a suspension of methyl 6-(6-Methyl-pyridin-3-ylamino)-nicotinate (2.72 g, 11.18 mmol) in methanol (55 mL) is added 2M NaOH (17 mL). The reaction mixture is heated to 60° C. for 30 min. After 15 min a clear reddish solution is formed. Then, the reaction flask is placed in an ice bath and 2M HCl (17 mL) is added at such a rate that the internal temperature does not exceed 20° C. After evaporation of methanol the suspension is diluted with water (50 mL). The product is filtered off, washed with cold water and vacuum dried at 60° C. over night to give a pink powder (2.78 g, 100%).

Following the same procedure, the following compounds can be prepared:

### EXAMPLE 4.2

### Azepan-1-yl-[6-(4-chloro-phenylamino)-pyridin-3-yl]-methanone

Yellow foam, TLC: R_{f}=0.25 (MTBE), HPLC: t_{R}=6.5 min (system 1); ESI+MS: m/z=330.5 (MH⁺).

### EXAMPLE 4.3

### [6-(4-Chloro-phenylamino)-pyridin-3-yl]-(3,3-difluoro-piperidin-1-yl)-methanone

Colorless crystals, TLC: R_{f}=0.23 (MTBE), HPLC: t_{R}=6.1 min (system 1); ESI+MS: m/z=352.6 (MH⁺).

### EXAMPLE 4.4

### [6-(4-Chloro-phenylamino)-pyridin-3-yl]-(4-methyl-piperidin-1-yl)-methanone

Colorless crystals, TLC: R_{f}=0.3 (MTBE), HPLC: t_{R}=6.6 min (system 1); ESI+MS: m/z=330.6 (MH⁺).

### EXAMPLE 4.5

### [6-(4-Chloro-phenylamino)-pyridin-3-yl]-(3,5-dimethyl-piperidin-1-yl)-methanone (diastereomeric mixture cis/trans 72:28)

Colorless crystals, TLC: R_{f}=0.35 (MTBE), HPLC: t_{R}=6.9 min (trans diastereomer, 28%), 7.0 min (cis diastereomer, 72%) (system 1); ESI+MS: m/z=344.6 (MH⁺).

### EXAMPLE 4.6

### rac-[6-(4-Chloro-phenylamino)-pyridin-3-yl]-(3-hydroxymethyl-piperidin-1-yl)-methanone

Colorless foam, TLC: R_{f}=0.32 (MTBE-MeOH 9:1), HPLC: t_{R}=5.2 min (system 1); ESI+MS: m/z=346.5 (MH⁺).

### EXAMPLE 4.7

### rac-[6-(4-Chloro-phenylamino)-pyridin-3-yl]-(3-methoxy-piperidin-1-yl)-methanone

Colorless foam, TLC: R_{f}=0.43 (MTBE-MeOH 9:1), HPLC: t_{R}=5.8 min (system 1); ESI+MS: m/z=346.5 (MH⁺).

### EXAMPLE 4.8

### [6-(4-Chloro-phenylamino)-pyridin-3-yl]-(octahydro-quinolin-1-yl)-meth anone (diastereomeric mixture, cis/trans)

Colorless foam, TLC: R_{f}=0.22, 0.29 (MTBE-MeOH 9:1), HPLC: t_{R}=7.3 min (system 1); ESI+MS: m/z=370.7 (MH⁺).

### EXAMPLE 4.9

### (3-Aza-bicyclo[3.2.2]non-3-yl)-[6-(4-chloro-phenylamino)-pyridin-3-yl]-methanone

Foam, TLC: R_{f}=0.28 (MTBE), HPLC: t_{R}=6.9 min (system 1); ESI+MS: m/z=356.6 (MH⁺).

### EXAMPLE 4.10

### (2-Aza-tricyclo[3.3.1.1*3,7*]dec-2-yl)-[6-(4-chloro-phenylamino)-pyridin-3-yl]-methanone

Colorless crystals, TLC: R_{f}=0.23 (MTBE), HPLC: t_{R}=7.0 min (system 1); ESI+MS: m/z=368.6 (MH⁺).

### EXAMPLE 4.11

### [6-(4-Chloro-phenylamino)-pyridin-3-yl]-(3-hydroxy-8-aza-bicyclo[3.2.1]oct-8-yl)-methanone

Colorless foam, TLC: R_{f}=0.36 (MTBE-MeOH 9:1), HPLC: t_{R}=5.4 min (system 1); ESI+MS: m/z=358.6 (MH⁺).

### EXAMPLE 4.12

### rac-(2-Aza-bicyclo[2.2.1]hept-2-yl)-[6-(4-chloro-phenylamino)-pyridin-3-yl]-methanone

Colorless crystals, TLC: R_{f}=0.31 (MTBE-MeOH 95:5), HPLC: t_{R}=6.2 min (system 1); ESI+MS: m/z=328.6 (MH⁺).

### EXAMPLE 4.13

### rac-(3-Methyl-piperidin-1-yl)-[6-(6-methyl-pyridin-3-ylamino)-pyridin-3-yl]-methanone

Yellow foam, TLC: R_{f}=0.26 (MTBE-MeOH 9:1), HPLC: t_{R}=4.4 min (system 1); ESI+MS: m/z=311.6 (MH⁺).

Using either S-3-methylpiperidine or R-3-methylpiperidine as starting material the pure enantiomers could be prepared:

### EXAMPLE 4.13a

### (S-3-Methyl-piperidin-1-yl)-[6-(6-methyl-pyridin-3-ylamino)-pyridin-3-yl]-methanone

Colorless foam, TLC: R_{f}=0.32 (MTBE-MeOH 85:15), HPLC: t_{R}=4.1 min (system 1); ESI+MS: m/z=311.2 (MH⁺).

### EXAMPLE 4.13b

### (R-3-Methyl-piperidin-1-yl)-[6-(6-methyl-pyridin-3-ylamino)-pyridin-3-yl]-methanone

Colorless foam, HPLC: t_{R}=4.1 min (system 1); ESI+MS: m/z=311.2 (MH⁺).

### EXAMPLE 4.14

### [6-(4-Chloro-phenylamino)-pyridin-3-yl]-(rel-(3aR,4S,7aR)-4-hydroxy-4-m-tolylethynyl-octahydro-indol-1-yl)-methanone

Yellow foam, TLC: R_{f}=0.32 (MTBE-MeOH 95:5), HPLC: t_{R}=6.8 min (system 1); ESI+MS: m/z=486.7 (MH⁺).

### EXAMPLE 4.15

### Azepan-1-yl-[6-(6-methyl-pyridin-3-ylamino)-pyridin-3-yl]-methanone

Yellow crystals, TLC: R_{f}=0.2 (MTBE-MeOH 9:1), HPLC: t_{R}=4.0 min (system 1); ESI+MS: m/z=311.6 (MH⁺).

### EXAMPLE 4.16

### Azocan-1-yl-[6-(6-methyl-pyridin-3-ylamino)-pyridin-3-yl]-methanone

TLC: R_{f}=0.33 (MTBE-MeOH 85:15), HPLC: t_{R}=4.5 min (system 1); ESI+MS: m/z=325.6 (MH⁺).

### EXAMPLE 4.17

### [5-Chloro-6-(6-methyl-pyridin-3-ylamino)-pyridin-3-yl]-(2-ethyl-piperidin-1-yl)-methanone

TLC: R_{f}=0.13 (DCM/MeOH 95:5), HPLC: t_{R}=2.8 min (system 4); LC/MS MS: m/z=359 (MH⁺).

### EXAMPLE 4.18

### [5-Chloro-6-(6-methyl-pyridin-3-ylamino)-pyridin-3-yl]-((R)-2-ethyl-piperidin-1-yl)-methanone

TLC: R_{f}=0.81 (DCM/MeOH 5:1), HPLC: t_{R}=2.8 min (system 4); LC/MS MS: m/z=359 (MH⁺); [α]_{D}=−33.6° (c=1.0, CHCl₃, 20° C.).

### EXAMPLE 4.19

### [5-Chloro-6-(6-methyl-pyridin-3-ylamino)-pyridin-3-yl]-((S)-2-ethyl-piperidin-1-yl)-methanone

TLC: R_{f}=0.81 (DCM/MeOH 95:5), HPLC: t_{R}=2.8 min (system 4); LC/MS MS: m/z=359 (MH⁺), [α]_{Hg578}=+1.64° (c=0.16, DCM, 20° C.).

### EXAMPLE 4.20

### [5-Chloro-6-(6-methyl-pyridin-3-ylamino)-pyridin-3-yl]-(2,3-dimethyl-piperidin-1-yl)-methanone

TLC: R_{f}=0.75 (DCM/MeOH 5:1), HPLC: t_{R}=2.8 min (system 4); LC/MS MS: m/z=359 (MH⁺).

### EXAMPLE 4.21

### [5-Chloro-6-(6-methyl-pyridin-3-ylamino)-pyridin-3-yl]-((2S,3S)-2,3-dimethyl-piperidin-1-yl)-methanone

TLC: R_{f}=0.66 (DCM/MeOH 5:1), HPLC: t_{R}=2.76 min (system 4); LC/MS MS: m/z=359 (MH⁺), [α]_{Hg578}=+0.9° (c=0.11, DCM, 20° C.).

### EXAMPLE 4.22

### [5-Chloro-6-(6-methyl-pyridin-3-ylamino)-pyridin-3-yl]-((2R,3R)-2,3-dimethyl-piperidin-1-yl)-methanone

TLC: R_{f}=0.72 (DCM/MeOH 5:1), HPLC: t_{R}=2.76 min (system 4); LC/MS MS: m/z=359 (MH⁺), [α]_{Hg578}=−1.0° (c=0.11, DCM, 20° C.).

### EXAMPLE 4.23

### [5-Chloro-6-(6-methyl-pyridin-3-ylamino)-pyridin-3-yl]-((S)-2-methyl-piperidin-1-yl)-methanone

TLC: R_{f}=0.87 (DCM/MeOH 5:1), HPLC: t_{R}=2.65 min (system 4); LC/MS MS: m/z=345 (MH⁺), [α]_{Hg578}=+0.10° (c=0.67, DCM, 20° C.).

### EXAMPLE 4.24

### [5-Chloro-6-(6-methyl-pyridin-3-ylamino)-pyridin-3-yl]-((R)-2-methyl-piperidin-1-yl)-methanone

TLC: R_{f}=0.80 (DCM/MeOH 5:1), HPLC: t_{R}=2.65 min (system 4); LC/MS MS: m/z=345 (MH⁺), [α]_{Hg578}=−0.10° (c=0.67, DCM, 20° C.).

### EXAMPLE 4.25

### [5-Chloro-6-(6-methyl-pyridin-3-ylamino)-pyridin-3-yl]-(octahydro-[1]pyridin-1-yl)-methanone

TLC: R_{f}=0.79 (DCM/MeOH 5:1), HPLC: t_{R}=2.76 min (system 4); LC/MS MS: m/z=371 (MH⁺).

### EXAMPLE 4.26

### [5-Chloro-6-(6-methyl-pyridin-3-ylamino)-pyridin-3-yl]-(4aS,7aS)-octahydro-[1]pyridin-1-yl-methanone

TLC: R_{f}=0.64 (DCM/MeOH 5:1), HPLC: t_{R}=2.86 min (system 4); LC/MS MS: m/z=371 (MH⁺), [α]_{Hg578}=+0.12° (c=0.007, DCM, 20° C.).

### EXAMPLE 4.27

### [5-Chloro-6-(6-methyl-pyridin-3-ylamino)-pyridin-3-yl]-(4aR,7aR)-octahydro-[1]pyridin-1-yl-methanone

TLC: R_{f}=0.64 (DCM/MeOH 5:1), HPLC: t_{R}=2.84 min (system 4); LC/MS MS: m/z=371 (MH⁺), [α]_{Hg578}=−0.15° (c=0.007, DCM, 20° C.).

### EXAMPLE 4.28

### [5-Chloro-6-(6-methyl-pyridin-3-ylamino)-pyridin-3-yl]-(2-isopropyl-piperidin-1-yl)-methanone

TLC: R_{f}=0.76 (DCM/MeOH 5:1), HPLC: t_{R}=2.90 min (system 4); LC/MS MS: m/z=373 (MH⁺).

### EXAMPLE 4.29

### [5-Chloro-6-(6-methyl-pyridin-3-ylamino)-pyridin-3-yl]-((R)-2-isopropyl-piperidin-1-yl)-methanone

TLC: R_{f}=0.63 (DCM/MeOH 5:1), HPLC: t_{R}=2.88 min (system 4); LC/MS MS: m/z=373 (MH⁺), [α]_{Hg578}=+0.72° (c=0.09, DCM, 20° C.).

### EXAMPLE 4.30

### [5-Chloro-6-(6-methyl-pyridin-3-ylamino)-pyridin-3-yl]-((S)-2-isopropyl-piperidin-1-yl)-methanone

TLC: R_{f}=0.63 (DCM/MeOH 5:1), HPLC: t_{R}=2.89 min (system 4); LC/MS MS: m/z=373 (MH⁺), [α]_{Hg578}=−0.79° (c=0.09, DCM, 20° C.).

### EXAMPLE 4.31

### [5-Chloro-6-(6-methyl-pyridin-3-ylamino)-pyridin-3-yl]-((R)-3-ethyl-piperidin-1-yl)-methanone

TLC: R_{f}=0.30 (EtOAc/hexanes 1:1), HPLC: t_{R}=2.83 min (system 4); LC/MS MS: m/z=359 (MH⁺).

### EXAMPLE 4.32

### [5-Chloro-6-(6-methyl-pyridin-3-ylamino)-pyridin-3-yl]-((S)-3-ethyl-piperidin-1-yl)-methanone

TLC: R_{f}=0.28 (EtOAc/hexanes 1:1), HPLC: t_{R}=2.86 min (system 4); LC/MS MS: m/z=359 (MH⁺).

### EXAMPLE 4.33

### [5-Chloro-6-(6-methyl-pyridin-3-ylamino)-pyridin-3-yl]-(3-cyclopropyl-piperidin-1-yl)-methanone

TLC: R_{f}=0.51 (DCM/MeOH 5:1), HPLC: t_{R}=2.90 min (system 4); LC/MS MS: m/z=373 (MH⁺).

The starting material can be prepared as described hereafter:

i) 3-Cyclopropyl-piperidine hydrochloride

3-Cyclopropyl pyridin (820 mg, 5.27 mmol) was hydrogenated in a mixture of MeOH (15 mL) and concentrated aqueous hydrochloric acid (0.58 mL) in the presence of Nishimura catalyst (70 mg) under atmospheric pressure for 22 hours. The mixture was filtered through a pad of celite and washed with MeOH. The solvent was removed in vacuo and the residue was dissolved in water. The aqueous solution was first washed with DCM, than basified by addition of 40% NaOH solution and extracted twice with DCM. The organic phases were combined, dried over sodium sulfate, acidified by addition of ethanolic hydrochloric acid, and concentrated in vacuo to afford 3-cyclopropyl piperidine hydrochloride (694 mg, 82%) as colorless crystals. TLC: R_{f}=0.49 (DCM/MeOH 5:1), LC/MS MS: m/z=126 (MH⁺).

### EXAMPLE 4.34

### [5-Chloro-6-(6-methyl-pyridin-3-ylamino)-pyridin-3-yl]-(2-propyl-piperidin-1-yl)-methanone

TLC: R_{f}=0.84 (DCM/MeOH 5:1), HPLC: t_{R}=2.95 min (system 4); LC/MS MS: m/z=373 (MH⁺).

### EXAMPLE 4.35

### [5-Chloro-6-(6-methyl-pyridin-3-ylamino)-pyridin-3-yl]-((S)-2-propyl-piperidin-1-yl)-methanone

TLC: R_{f}=0.69 (DCM/MeOH 5:1), HPLC: t_{R}=2.97 min (system 4); LC/MS MS: m/z=373 (MH⁺), [α]_{Hg578}=+1.17° (c=0.09, DCM, 20° C.).

### EXAMPLE 4.36

### [5-Chloro-6-(6-methyl-pyridin-3-ylamino)-pyridin-3-yl]-((R)-2-propyl-piperidin-1-yl)-methanone

TLC: R_{f}=0.61 (DCM/MeOH 5:1), HPLC: t_{R}=2.97 min (system 4); LC/MS MS: m/z=373 (MH⁺), [α]_{Hg578}=−1.17° (c=0.09, DCM, 20° C.).

### EXAMPLE 4.37

### [5-Chloro-6-(6-methyl-pyridin-3-ylamino)-pyridin-3-yl]-(2,3-diethyl-piperidin-1-yl)-methanone

TLC: R_{f}=0.21 (EtOAc/hexanes 1:1), HPLC: t_{R}=3.08 min (system 4); LC/MS MS: m/z=387 (MH⁺).

### EXAMPLE 4.38

### (2-Butyl-piperidin-1-yl)-[5-chloro-6(6-methyl-pyridin-3-ylamino)-pyridin-3-yl]-methanone

TLC: R_{f}=0.22 (DCM/MeOH 5:1), HPLC: t_{R}=3.09 min (system 4); LC/MS MS: m/z=387 (MH⁺).

### EXAMPLE 4.39

### [5-Chloro-6-(6-methyl-pyridin-3-ylamino)-pyridin-3-yl]-[2-(1-ethyl-propyl)-piperidin-1-yl]-methanone

TLC: R_{f}=0.87 (DCM/MeOH 95:5), HPLC: t_{R}=3.19 min (system 4); LC/MS MS: m/z=401 (MH⁺).

### EXAMPLE 4.40

### [5-Chloro-6-(6-methyl-pyridin-3-ylamino)-pyridin-3-yl]-(2-ethyl-3-methyl-piperidin-1-yl)-methanone

TLC: R_{f}=0.22 (EtOAc/hexanes 3:1), HPLC: t_{R}=2.89 min (system 4); LC/MS MS: m/z=373 (MH⁺).

The starting material was prepared as described hereafter:

i) 2-Ethyl-3-methyl-pyridin

2-Ethyl-3-methylpyridin was prepared by Suzuki coupling of 2-bromo-3-methylpyridin and ethylboronic acid according to the procedure given in Tetrahedron Letters 2002, 43, 6987-6990. The desired product was obtained in 52% yield after purification on silica gel.

ii) 2-Ethyl-3-methyl-piperidine hydrochloride

2-Ethyl-3-methylpyridin (1.75 g, 11.1 mmol) was hydrogenated in a mixture of MeOH (32 mL) and concentrated aqueous hydrochloric acid (1.2 mL) in the presence of Nishimura catalyst (180 mg) under atmospheric pressure for 22 hours. The mixture was filtered through a pad of celite and washed with MeOH. The solvent was removed in vacuo and the residue was dissolved in water. The aqueous solution was first washed with DCM, than basified by addition of 40% NaOH solution and extracted twice with DCM. The organic phases were combined, dried over sodium sulfate, acidified by addition of ethanolic hydrochloric acid, and concentrated in vacuo to afford 2-ethyl-3-methyl piperidine hydrochloride (1.60 g, 88%) as colorless crystals.

### EXAMPLE 4.41

### [5-Chloro-6-(6-methyl-pyridin-3-ylamino)-pyridin-3-yl]-(2-phenyl-piperidin-1-yl)-methanone

TLC: R_{f}=0.34 (EtOAc/hexanes 3:1), HPLC: t_{R}=1.85 min (system 5); LC/MS MS: m/z=407 (MH⁺).

### EXAMPLE 4.42

### [5-Chloro-6-(6-methyl-pyridin-3-ylamino)-pyridin-3-yl]-(3,4,5,6-tetrahydro-2H-[2,2′]bipyridinyl-1-yl)-methanone

TLC: R_{f}=0.17 (EtOAc/hexanes 3:1), HPLC: t_{R}=2.29 min (system 5); LC/MS MS: m/z=408 (MH⁺).

### EXAMPLE 4.43

### [5-Chloro-6-(6-methyl-pyridin-3-ylamino)-pyridin-3-yl]-(3,4,5,6-tetrahydro-2H-[2,3′]bipyridinyl-1-yl)-methanone

TLC: R_{f}=0.25 (DCM/MeOH 9:1), HPLC: t_{R}=2.11 min (system 5); LC/MS MS: m/z=408 (MH⁺).

### EXAMPLE 4.44

### [5-Chloro-6-(6-methyl-pyridin-3-ylamino)-pyridin-3-yl]-[2-(tetrahydro-furan-2-yl)-piperidin-1-yl]-methanone

TLC: R_{f}=0.34 (DCM/MeOH 9:1), HPLC: t_{R}=2.62 min (system 4); LC/MS MS: m/z=401 (MH⁺).

### EXAMPLE 4.45

### [5-Chloro-6-(6-methyl-pyridin-3-ylamino)-pyridin-3-yl]-[2-(5-methyl-furan-2-yl)-piperidin-1-yl]-methanone

TLC: R_{f}=0.63 (DCM/MeOH 9:1), HPLC: t_{R}=3.00 min (system 4); LC/MS MS: m/z=411 (MH⁺).

### EXAMPLE 4.46

### [5-Chloro-6-(6-methyl-pyridin-3-ylamino)-pyridin-3-yl]-(2-oxazol-2-yl-piperidin-1-yl)-methanone

TLC: R_{f}=0.55 (DCM/MeOH 9:1), HPLC: t_{R}=2.66 min (system 4); LC/MS MS: m/z=414 (MH⁺).

### EXAMPLE 4.47

### [2-(2-Chloro-ethyl)-piperidin-1-yl]-[5-chloro-6-(6-methyl-pyridin-3-ylamino)-pyridin-3-yl]-methanone

TLC: R_{f}=0.33 (DCM/MeOH 5:1), HPLC: t_{R}=0.76 min (system 4); LC/MS MS: m/z=394 (MH⁺).

### EXAMPLE 4.48

### [5-Chloro-6-(6-methyl-pyridin-3-ylamino)-pyridin-3-yl]-(2,6-dimethyl-piperidin-1-yl)-methanone

TLC: R_{f}=0.25 (EtOAc/hexanes 3:1), HPLC: t_{R}=3.03 min (system 3); LC/MS MS: m/z=359 (MH⁺).

### EXAMPLE 4.49

### [5-Chloro-6-(6-methyl-pyridin-3-ylamino)-pyridin-3-yl]-(2, 2,6,6-tetramethyl-piperidin-1-yl)-methanone

TLC: R_{f}=0.44 (EtOAc/hexanes 3:1), HPLC: t_{R}=3.14 min (system 4); LC/MS MS: m/z=387 (MH⁺).

### EXAMPLE 4.50

### [5-Chloro-6-(6-methyl-pyridin-3-ylamino)-pyridin-3-yl]-(2-methyl-6-propyl-piperidin-1-yl)-methanone

TLC: R_{f}=0.35 (EtOAc/hexanes 3:1), HPLC: t_{R}=2.13 min (system 5); LC/MS MS: m/z=387 (MH⁺).

### EXAMPLE 4.51

### [5-Chloro-6-(6-methyl-pyridin-3-ylamino)-pyridin-3-yl]-((2R,6R)-2-ethyl-6-propyl-piperidin-1-yl)-methanone

TLC: R_{f}=0.45 (EtOAc/hexanes 3:1), HPLC: t_{R}=2.25 min (system 5); LC/MS MS: m/z=387 (MH⁺).

### EXAMPLE 4.52

### [5-Chloro-6-(6-methyl-pyridin-3-ylamino)-pyridin-3-yl]-(5-methyl-2-propyl-piperidin-1-yl)-methanone

TLC: R_{f}=0.40 (EtOAc/hexanes 3:1), HPLC: t_{R}=2.06 min (system 5); LC/MS MS: m/z=387 (MH⁺).

The starting material was prepared as described hereafter:

i) 5-Methyl-2-propyl-pyridin

5-Methyl-2-propyl-pyridin was prepared by Suzuki coupling of 2-bromo-5-methylpyridin and propylboronic acid according to the procedure given in Tetrahedron Letters 2002, 43, 6987-6990. The desired product was obtained in 24% yield after purification on silica gel.

ii) 5-Methyl-2-propyl-piperidine hydrochloride

5-Methyl-2-propyl-pyridin (345 mg, 2.55 mmol) was hydrogenated in a mixture of MeOH (10 mL) and concentrated aqueous hydrochloric acid (0.29 mL) in the presence of Nishimura catalyst (50 mg) under atmospheric pressure for 40 hours. The mixture was filtered through a pad of celite and washed with MeOH. The solvent was removed in vacuo and the residue was dissolved in water. The aqueous solution was first washed with DCM, than basified by addition of 40% NaOH solution and extracted twice with DCM. The organic phases were combined, dried over sodium sulfate, acidified by addition of ethanolic hydrochloric acid, and concentrated in vacuo to afford 2-methyl-3-propyl piperidine hydrochloride (0.43 g, 95%) as beige crystals.

### EXAMPLE 4.53

### [5-Chloro-6-(4-chloro-phenylamino)-pyridin-3-yl]-(octahydro-1]pyridin-1-yl)-methanone

TLC: R_{f}=0.71 (DCM/MeOH 95:5), HPLC: t_{R}=3.85 min (system 5); LC/MS MS: m/z=391 (MH⁺).

### EXAMPLE 4.54

### [5-Chloro-6-(4-chloro-phenylamino)-pyridin-3-yl]-((R)-2-ethyl-piperidin-1-yl)-methanone

TLC: R_{f}=0.75 (DCM/MeOH 95:5), HPLC: t_{R}=3.78 min (system 5); LC/MS MS: m/z=379 (MH⁺).

### EXAMPLE 4.55

### [5-Chloro-6-(6-methyl-pyridin-3-ylamino)-pyridin-3-yl]-(2-vinyl-piperidin-1-yl)-methanone

TLC: R_{f}=0.78 (DCM/MeOH 95:5), HPLC: t_{R}=2.70 min (system 4); LC/MS MS: m/z=357 (MH⁺).

### EXAMPLE 4.56

### [5-Chloro-6-(6-methyl-pyridin-3-ylamino)-pyridin-3-yl]-[((Z)-2-propenyl)-piperidin-1-yl]-methanone

TLC: R_{f}=0.66 (DCM/MeOH 95:5), HPLC: t_{R}=2.88 min (system 4); LC/MS MS: m/z=371 (MH⁺).

### EXAMPLE 4.57

### [5-Chloro-6-(6-methyl-pyridin-3-ylamino)-pyridin-3-yl]-[3-ethylidene-piperidin-1-yl]-methanone

TLC: R_{f}=0.89 (DCM/MeOH 5:1), HPLC: t_{R}=2.85 min (system 4); LC/MS MS: m/z=357 (MH⁺).

The starting material was prepared as described hereafter:

i) 3-Ethylidene-piperidine hydrochloride

To a solution of potassium-tert-butoxide (3.10 g, 27.6 mmol, 1.1 eq) in THF (30 mL) at rt was added sequentially ethyltriphenylphosphoniumbromide (11.0 g, 29.6 mmol, 1.18 eq) followed by a solution of 1-(tert-Butoxycarbonyl)-3-piperidone (5.0 g, 25.1 mmol) in THF (20 mL). After stirring the resulting suspension for 24 h at rt, water was added and the aqueous phase was extracted with DCM. The organic phases were combined, dried over sodium sulfate and the solvent removed on vacuo. After purification by flash chromatography, 3-ethylidene-piperidine-1-carboxylic acid tert-butyl ester (5.5 g, 100%) was obtained as a 1:2 E/Z isomeric mixture. Deprotection of the Boc-group was effected by stirring 3-ethylidene-piperidine-1-carboxylic acid tert-butyl ester (5.5 g, 26 mmol) in HCl/dioxane (4M, 15 mL) for 1 h at rt. The white precipitate was filtered off, washed twice with diethyl ether and dried on vacuo to afford the desired product as beige crystals (2.99 g, 78%). LC/MS MS: m/z=111 (MH⁺).

### EXAMPLE 4.58

### [5-Chloro-6-(6-methyl-pyridin-3-ylamino)-pyridin-3-yl]-[3-propylidene-piperidin-1-yl]-methanone

TLC: R_{f}=0.16 (DCM/MeOH 95:5), HPLC: t_{R}=2.85 min (system 4); LC/MS MS: m/z=371 (MH⁺).

3-Propylidene-piperidine hydrochloride was prepared in an overall yield of 71% starting from propyltriphenylphosphoniumbromide and 1-(tert-Butoxycarbonyl)-3-piperidone in analogy to the procedure given in Example 4.57 i. LC/MS MS: m/z=126 (MH⁺).

### EXAMPLE 4.59

### [5-Chloro-6-(6-methyl-pyridin-3-ylamino)-pyridin-3-yl]-(2-ethoxymethyl-piperidin-1-yl)-methanone

TLC: R_{f}=0.66 (DCM/MeOH 95:5), HPLC: t_{R}=2.88 min (system 4); LC/MS MS: m/z=371 (MH⁺).

### EXAMPLE 4.60

### [5-Chloro-6-(6-methyl-pyridin-3-ylamino)-pyridin-3-yl]-(2-ethoxymethyl-piperidin-1-yl)-methanone

TLC: R_{f}=0.26 (DCM/MeOH 95:5), HPLC: t_{R}=2.55 min (system 4); LC/MS MS: m/z=389 (MH⁺).

### EXAMPLE 4.61

### [5-Chloro-6(6-methyl-pyridin-3-ylamino)-pyridin-3-yl]-[2-(2-hydroxy-ethyl)-piperidin-1-yl]-methanone

TLC: R_{f}=0.23 (DCM/MeOH 95:5), HPLC: t_{R}=2.72 min (system 4); LC/MS MS: m/z=375 (MH⁺).

### EXAMPLE 4.62

### [5-Chloro-6-(6-methyl-pyridin-3-ylamino)-pyridin-3-yl]-(5-fluoro-2-propyl-piperidin-1-yl)-methanone

TLC: R_{f}=0.35 (EtOAc/hexanes 2:1), HPLC: t_{R}=1.60 min (system 5); LC/MS MS: m/z=391 (MH⁺).

The starting material was prepared as described hereafter:

i) 5-Fluoro-2-propyl pyridin

To a suspension of n-propylmagnesium chloride (13 mL, 1.0 M in diethyl ether) and zinc chloride (17 mL, 0.5 M in THF, 2.5 eq) was added 1-methyl-2-pyrrolidinone (10 mL), 2-bromo-5-fluoropyridin (600 mg, 3.41 mmol) and bis(tri-tert.-butylphosphine)palladium (174 mg, 0.34 mmol, 0.1 eq). After stirring at 80° C. for 3 h the mixture was cooled to 0° C., water was added resulting solution extracted with EtOAc twice. The organic phases were combined, dried over sodium sulfate and the solvent removed on vacuo. After purification by flash chromatography, 5-fluoro-2-propyl pyridin (182 mg, 30%) was obtained. LC/MS MS: m/z=140 (MH⁺).

ii) 5-Fluoro-2-propyl-piperidine hydrochloride

5-Fluoro-2-propyl pyridin (182 mg, 1.04 mmol) was hydrogenated in a mixture of MeOH (10 mL) and concentrated aqueous hydrochloric acid (0.13 mL) in the presence of Nishimura catalyst (50 mg) at 4 bar for 3.5 hours. The mixture was filtered through a pad of celite and washed with MeOH. The solvent was removed in vacuo and the residue was dissolved in water. The aqueous solution was first washed with DCM, than basified by addition of 40% NaOH solution and extracted twice with DCM. The organic phases were combined, dried over sodium sulfate, acidified by addition of ethanolic hydrochloric acid, and concentrated in vacuo to afford a mixture of 5-fluoro-2-propyl-piperidine hydrochloride and 2-propyl-piperidine hydrochloride as light red solid (95%) which was used in the next step without further purification.

### EXAMPLE 4.63

### [5-Chloro-6-(6-methyl-pyridin-3-ylamino)-pyridin-3-yl]-[2-(1,2-difluoro-propyl)-piperidin-1-yl]-methanone and

### EXAMPLE 4.64

### [5-Chloro-6-(6-methyl-pyridin-3-ylamino)-pyridin-3-yl]-[2-(2-fluoro-propyl)-piperidin-1-yl]-methanone

Both compounds were isolated after preparative TLC separation of the corresponding mixture.

[5-Chloro-6-(6-methyl-pyridin-3-ylamino)-pyridin-3-yl]-[2-(1,2-difluoro-propyl)-piperidin-1-yl]-methanone: TLC: R_{f}=0.39 (EtOAc/hexanes 5:1), HPLC: t_{R}=1.37 min (system 5); LC/MS MS: m/z=391 (MH⁺).

[5-Chloro-6-(6-methyl-pyridin-3-ylamino)-pyridin-3-yl]-[2-(1-fluoro-propyl)-piperidin-1-yl]-methanone: TLC: R_{f}=0.40 (EtOAc/hexanes 5:1), HPLC: t_{R}=1.22 min (system 5); LC/MS MS: m/z=409 (MH⁺).

The starting material was prepared as described hereafter:

i) 2-(1,2-Difluoro-propenyl)-pyridin

To a solution of 1-pyridin-2-yl-propan-2-one (3.75 g, 27.7 mmol) in DCM (20 mL) at 0° C. was was DAST (10.1 mL, 69 mmol, 2.50 eq). After stirring the solution for 15 h (0° C.→rt) it was diluted by DCM and subsequently quenched by slow addition of ice water. Resulting solution was extracted twice with DCM. The organic phases were combined, dried over sodium sulfate and the solvent removed on vacuo. After purification by flash chromatography 2-(1,2-difluoro-propenyl)-pyridin (616 mg, 14%) was obtained as beige oil. LC/MS MS: m/z=156 (MH⁺).

ii) 2-(1,2-Difluoro-propyl)-piperidine hydrochloride, 2-(1-fluoro-propyl)-piperidine hydrochloride and 2-propyl-piperidine hydrochloride

2-(1,2-Difluoro-propenyl)-pyridin (820 mg, 4.28 mmol) was hydrogenated in a mixture of MeOH (25 mL) and concentrated aqueous hydrochloric acid (0.46 mL) in the presence of Nishimura catalyst (100 mg) at atmospheric pressure for 24 hours. The mixture was filtered through a pad of celite and washed with MeOH. The solvent was removed in vacuo and the residue was dissolved in water. The aqueous solution was first washed with DCM, than basified by addition of 40% NaOH solution and extracted twice with DCM. The organic phases were combined, dried over sodium sulfate, acidified by addition of ethanolic hydrochloric acid, and concentrated in vacuo to afford a mixture of 2-(1,2-difluoro-propyl)-piperidine hydrochloride, 2-(1-fluoro-propyl)-piperidine hydrochloride and 2-propyl-piperidine hydrochloride as light red solid (100%) which was used in the next step without further purification.

### EXAMPLE 4.65

### [5-Chloro-6-(6-methyl-pyridin-3-ylamino)-pyridin-3-yl]-(2-ethyl-[1,3]oxazepan-3-yl)-methanone

TLC: R_{f}=0.21 (DCM/MeOH 95:5), HPLC: t_{R}=2.66 min (system 4); LC/MS MS: m/z=375 (MH⁺).

### EXAMPLE 4.66

### [5-Chloro-6-(6-methyl-pyridin-3-ylamino)-pyridin-3-yl]-(2-propyl-[1,3]oxazepan-3-yl)-methanone

TLC: R_{f}=0.2 (DCM/MeOH 95:5), HPLC: t_{R}=2.82 min (system 4); LC/MS MS: m/z=389 (MH⁺).

### EXAMPLE 4.67

### [5-Chloro-6-(6-methyl-pyridin-3-ylamino)-pyridin-3-yl]-[2-(1-ethyl-propyl)-[1,3]oxazepan-3-yl]-methanone

TLC: R_{f}=0.17 (DCM/MeOH 95:5), HPLC: t_{R}=3.10 min (system 4); LC/MS MS: m/z=417 (MH⁺).

### EXAMPLE 4.68

### [5-Chloro-6-(6-methyl-pyridin-3-ylamino)-pyridin-3-yl]-(2-propyl-[1,3]oxazinan-3-yl)-methanone

TLC: R_{f}=0.11 (DCM/MeOH 95:5), HPLC: t_{R}=2.55 min (system 4); LC/MS MS: m/z=375 (MH⁺).

### EXAMPLE 4.69

### [5-Chloro-6-(6-methyl-pyridin-3-ylamino)-pyridin-3-yl]-[2-(1-ethyl-propyl)-[1,3]oxazinan-3-yl]-methanone

TLC: R_{f}=0.18 (DCM/MeOH 95:5), HPLC: t_{R}=2.89 min (system 4); LC/MS MS: m/z=403 (MH⁺).

### EXAMPLE 4.70

### (2-Butyl-[1,3]oxazinan-3-yl)-[5-chloro-6-(6-methyl-pyridin-3-ylamino)-pyridin-3-yl]-methanone

TLC: R_{f}=0.12 (DCM/MeOH 95:5), HPLC: t_{R}=2.87 min (system 4); LC/MS MS: m/z=389 (MH⁺).

### EXAMPLE 4.71

### [5-Chloro-6-(6-methyl-pyridin-3-ylamino)-pyridin-3-yl]-[2-(2-methoxy-ethyl)-piperidin-1-yl]-methanone

TLC: R_{f}=0.15 (DCM/MeOH 95:5), HPLC: t_{R}=2.60 min (system 4); LC/MS MS: m/z=389 (MH⁺).

### EXAMPLE 4.72

### [5-Chloro-6-(6-methyl-pyridin-3-ylamino)-pyridin-3-yl]-(2-phenyl-pyrrolidin-1-yl)-methanone

TLC: R_{f}=0.66 (DCM/MeOH 9:1), HPLC: t_{R}=3.07 min (system 3); LC/MS MS: m/z=393 (MH⁺).

### EXAMPLE 4.73

### [5-Chloro-6-(6-methyl-pyridin-3-ylamino)-pyridin-3-yl]-(2-pyridin-2-yl-pyrrolidin-1-yl)-methanone

TLC: R_{f}=0.65 (DCM/MeOH 9:1), HPLC: t_{R}=2.48 min (system 3); LC/MS MS: m/z=394 (MH⁺).

### EXAMPLE 4.74

### [5-Chloro-6-(6-methyl-pyridin-3-ylamino)-pyridin-3-yl]-((R)-2-ethoxy-pyrrolidin-1-yl)-methanone

TLC: R_{f}=0.44 (DCM/MeOH 9:1), HPLC: t_{R}=2.09 min (system 3); LC/MS MS: m/z=361 (MH⁺).

### EXAMPLE 4.75

### [5-Chloro-6-(6-methyl-pyridin-3-ylamino)-pyridin-3-yl]-[2-(5-methyl-thiophen-2-yl)-pyrrolidin-1-yl]-methanone

TLC: R_{f}=0.5 (DCM/MeOH 9:1), HPLC: t_{R}=3.15 min (system 3); LC/MS MS: m/z=413 (MH⁺).

### EXAMPLE 4.76

### [5-Chloro-6-(6-methyl-pyridin-3-ylamino)-pyridin-3-yl]-(2-propyl-azepan-1-yl)-methanone

TLC: R_{f}=0.52 (DCM/MeOH 95:5), HPLC: t_{R}=3.21 min (system 3); LC/MS MS: m/z=387 (MH⁺).

### EXAMPLE 4.77

### [5-Chloro-6-(6-methyl-pyridin-3-ylamino)-pyridin-3-yl]-(3-propyl-morpholin-4-yl)-methanone

TLC: R_{f}=0.18 (DCM/MeOH 95:5), HPLC: t_{R}=2.53 min (system 4); LC/MS MS: m/z=375 (MH⁺).

### EXAMPLE 5.1

### Azepan-1-yl-[5-chloro-6-(4-chloro-phenylamino)-pyridin-3-yl]-methanone

To a solution of 5-Chloro-6-(4-chloro-phenylamino)nicotinic acid (72 mg, 0.25 mmol) and DIPEA (67 μL, 0.38 mmol) in 1,2-dimethoxyethane (1.2 mL) is added HATU (97 mg, 0.25 mmol) in one portion. The reaction mixture is stirred for 30 min at RT. Then, hexamethylene imine (24 μL, 0.2 mmol) is injected and stirring is continued for further 6 h. The reaction mixture is evaporated to dryness and the residue is purified by preparative HPLC(YMC Pack Pro C₁₈ 5 μm, 150×30 mm; AcN-H₂O-0.1% TFA gradient 10%->100% AcN; flow: 20 mL min⁻¹). The fractions containing the product are combined and acetonitrile is evaporated. The remaining aqueous solution is made alkaline by addition of solid NaHCO₃ and extracted with ethyl acetate. The organic layer is separated, washed with brine, dried over Na₂SO₄, and evaporated to dryness to afford the title compound as a colorless powder (75 mg, 81%), HPLC: t_{R}=7.0 min (system 1); ESI+MS: m/z=364.0, 366.0 (MH⁺).

The starting material can be prepared as described hereafter:

5-Chloro-6-(4-chloro-phenylamino)-nicotinic acid

A solution of 5,6-dichloronicotinic acid (0.5 g, 2.55 mmol) and 4-chloroaniline (293 mg, 2.30 mmol) in glacial acetic acid (5 mL) is microwave heated to 150° C. for 75 min. To the clear solution is added ethyl acetate (10 mL). After a short time the product starts to crystallize. The precipitate is filtered off, washed with ethyl acetate, and vacuum dried at room temperature to afford the desired product as a colorless powder (470 mg, 65%).

Following the same procedure, the following compounds can be prepared:

### EXAMPLE 5.2

### rac-[5-Chloro-6-(4-chloro-phenylamino)-pyridin-3-yl]-(3-methyl-piperidin-1-yl)-methanone

Colorless syrup, HPLC: t_{R}=7.2 min (system 1); ESI+MS: m/z=364.0, 366.0 (MH⁺).

Using either S-3-methylpiperidine or R-3-methylpiperidine as starting material the pure enantiomers could be prepared:

### EXAMPLE 5.2a

### [5-Chloro-6-(4-chloro-phenylamino)-pyridin-3-yl]-(S-3-methyl-piperidin-1-yl)-methanone

Brown gum, HPLC: t_{R}=7.4 min (system 1); ESI+MS: m/z=364.0, 366.0 (MH⁺).

### EXAMPLE 5.2b

### [5-Chloro-6-(4-chloro-phenylamino)-pyridin-3-yl]-(R-3-methyl-piperidin-1-yl)-methanone

Brown gum, HPLC: t_{R}=7.3 min (system 1); ESI+MS: m/z=364.0, 366.0 (MH⁺).

### EXAMPLE 5.3

### Azepan-1-yl-[2-(4-chloro-phenylamino)-pyrimidin-5-yl]-methanone

Colorless crystals, HPLC: t_{R}=6.4 min (system 1); ESI+MS: m/z=331.5 (MH⁺).

### EXAMPLE 5.4

### [2-(4-Chloro-phenylamino)-pyrimidin-5-yl]-piperidin-1-yl-methanone

Colorless crystals, HPLC: t_{R}=6.2 min (system 1); ESI+MS: m/z=317.6 (MH⁺).

### EXAMPLE 5.5

### rac-[2-(4-Chloro-phenylamino)-pyrimidin-5-yl]-(3-methyl-piperidin-1-yl)-methanone

Colorless crystals, HPLC: t_{R}=6.5 min (system 1); ESI+MS: m/z=331.6 (MH⁺).

### EXAMPLE 6.1

### Azepan-1-yl-[6-(4-chloro-phenylamino)-5-methoxy-pyridin-3-yl]-methanone

To a solution of azepan-1-yl-(6-chloro-5-methoxy-pyridin-3-yl)-methanone (198 mg, 0.70 mmol) and 4-chloroaniline (270 mg, 2.11 mmol) in toluene (5 mL) is added finely ground anhydrous K₂CO₃ (491 mg, 3.52 mmol). To the suspension obtained is added a still warm solution prepared by dissolving palladium(II) acetate (10 mg, 0.04 mmol) and BINAP (27 mg, 0.04 mmol) in toluene (1 mL) with stirring for 20 min at 90° C. The reaction mixture is stirred under argon for 21 h at 80° C. After cooling ethyl acetate (40 mL) is added and the solution is extracted with water (3×15 mL). The organic layer is isolated, dried over Na₂SO₄ and evaporated to dryness to give a dark green oil. The crude product is purified by flash chromatography (24 g silica gel, MeOH-MTBE gradient 2%->15% MeOH, flow 20 mL min¹). Recrystallization from Et₂O gives the desired compound as beige crystals, TLC: R_{f}=0.14 (MTBE), HPLC: t_{R}=7.0 min (system 1); ESI+MS: m/z=360.1 (MH⁺).

The starting material can be prepared as described hereafter:

i) 6-chloro-5-methoxynicotinic acid

To a solution of methyl 6-chloro-5-hydroxynicotinate (0.95 g, 5.07 mmol, prepared according to WO 00/51614) in DMSO (9.5 mL) is added powdered 85% KOH (0.67 g, 10.1 mmol) followed by slow injection of methyl iodide (0.35 mL, 5.57 mmol). The reaction mixture is stirred over night at RT. To achieve complete hydrolysis of the intermediate ester water (1 mL) is added and stirring is continued for further 30 min. The solution is diluted with 1 M HCl (100 mL) and extracted with ethyl acetate (1×100 mL, 3×50 mL). The combined organic extracts are dried over Na₂SO₄ and evaporated to give a yellow solid residue. Trituration with H₂O (20 mL) followed by drying in vacuo at 65° C. affords the title compound as beige powder (846 mg, 89%).

ii) Azepan-1-yl-(6-chloro-5-methoxy-pyridin-3-yl)-methanone

A mixture of 6-chloro-5-methoxynicotinic acid (272 mg, 1.45 mmol) and thionyl chloride (3.2 mL) is stirred for 30 min at 75° C. The clear solution is evaporated to dryness and the residue is redissolved in DCM (4 mL) under argon. After the addition of triethylamin (2 mL, 14.5 mmol) and hexamethyleneamine (0.2 mL, 1.74 mmol) the yellow turbid reaction mixture is stirred for 1 h at RT. Then MTBE (30 mL) is added and the solution is extracted with H₂O (3×10 mL), dried over Na₂SO₄ and evaporated to give the title compound as a yellow oil (407 mg, 100%). The material can be used in the next step without further purification.

Following the same procedure, the following compounds can be obtained:

### EXAMPLE 6.2

### Azepan-1-yl-[5-methoxy-6-(6-methyl-pyridin-3-ylamino)-pyridin-3-yl]-methanone]

Yellow lyophilisate, TLC: R_{f}=0.16 (MTBE-MeOH 9:1), HPLC: t_{R}=4.5 min (system 1); ESI+MS: m/z=341.1 (MH⁺).

### EXAMPLE 6.3

### [6-(4-Chloro-phenylamino)-5-methoxy-pyridin-3-yl]-piperidin-1-yl-methanone

Yellowish crystals, TLC: R_{f}=0.13 (MTBE), HPLC: t_{R}=6.9 min (system 1); ESI+MS: m/z=346.1 (MH⁺).

### EXAMPLE 6.4

### [5-Methoxy-6-(6-methyl-pyridin-3-ylamino)-pyridin-3-yl]-piperidin-1-yl-methanone

Yellowish lyophilisate, TLC: R_{f}=0.15 (MTBE-MeOH 9:1), HPLC: t_{R}=4.0 min (system 1); ESI+MS: m/z=327.1 (MH⁺).

### EXAMPLE 6.5

### Azepan-1-yl-[6-(4-chloro-phenylamino)-5-ethoxy-pyridin-3-yl]-methanone

Colorless lyophilisate, TLC: R_{f}=0.29 (MTBE), HPLC: t_{R}=7.4 min (system 1); ESI+MS: m/z=374.1 (MH⁺).

### EXAMPLE 6.6

### Azepan-1-yl-[5-ethoxy-6-(6-methyl-pyridin-3-ylamino)-pyridin-3-yl]-methanone

Colorless lyophilisate, TLC: R_{f}=0.25 (MTBE-MeOH 9:1), HPLC: t_{R}=4.6 min (system 1); ESI+MS: m/z=355.2 (MH⁺).

### EXAMPLE 6.7

### [5-Ethoxy-6-(6-methyl-pyridin-3-ylamino)-pyridin-3-yl]-piperidin-1-yl-methanone

Colorless lyophilisate, TLC: R_{f}=0.21 (MTBE-MeOH 9:1), HPLC: t_{R}=4.4 min (system 1); ESI+MS: m/z=341.2 (MH⁺).

### EXAMPLE 7.1

### [5-Chloro-6-(6-chloro-pyridin-3-ylamino)-pyridin-3-yl]-(3-methyl-piperidin-1-yl)-methanone

A solution of 5-[3-Chloro-5-(3-methyl-piperidine-1-carbonyl)-pyridin-2-ylamino]-1H-pyridin-2-one (88 mg, 0.25 mmol) and DMAP (5 mg, 0.04 mmol) in phosphoryl chloride (2.75 mL) is refluxed under argon for 90 h. After cooling the suspension obtained is evaporated and taken up in DCM (40 mL)-20% KHCO₃ solution (40 mL). The organic layer is washed (1×20% KHCO₃, 40 mL; 2×H₂O, 20 mL), dried over Na₂SO₄ and evaporated to give a reddish turbid syrup. The crude material is purified by flash chromatography (25 g silica gel, eluent MTBE, flow 20 mL min⁻) to afford a bluish foam (37 mg, 40%), TLC: R_{f}=0.39 (MTBE), HPLC: t_{R}=6.4 min (system 1); ESI+MS: m/z=365.0 (MH⁺).

The starting material can be prepared as described hereafter:

5-[3-Chloro-5-(3-methyl-piperidine-1-carbonyl)-pyridin-2-ylamino]-1H-pyridin-2-one

To a solution of [5-Chloro-6-(6-methoxy-pyridin-3-ylamino)-pyridin-3-yl]-(3-methyl-piperidin-1-yl)-methanone (489 mg, 1.36 mmol, prepared from 5,6-dichloronicotinic acid, 3-methylpiperidine and 3-amino-6-methoxypyridin according to the procedure given in example 5.1) in 1,2-dichloroethane (30 mL) is added iodotrimethyl silane (0.47 mL, 3.39 mmol) in one portion. The reaction mixture is stirred for 6 h at 70° C. under argon. After cooling the reaction is quenched with methanol (3 mL) stirred for 15 min at RT and evaporated. The residue is taken up in a mixture of DCM (40 mL) and triethyl amine (1 mL), extracted (1×H₂O, 20 mL; 1×5% NaS₂O₃, 20 mL, 1×H₂O, 20 mL), dried over Na₂SO₄ and evaporated to afford greenish residue. The crude product is purified by flash chromatography (54 g silica gel, MeOH-DCM gradient 0%->10% MeOH, flow 40 mL min⁻¹) to afford a beige foam (401 mg, 85%).

### EXAMPLE 8.1

### [6-(4-Chloro-phenylamino)-pyridazin-3-yl]-piperidin-1-yl-methanone

To a solution of 6-(4-Chloro-phenylamino)-pyridazine-3-carboxylic acid (50 mg, 0.2 mmol) and DIPEA (53 μL, 0.3 mmol) in DMA (1 mL) is added HATU (76 mg, 0.2 mmol) in one portion. The reaction mixture is stirred for 30 min at RT. Then piperidine (16 uL, 0.16 mmol) is injected and stirring is continued for further 6 h. The solution is diluted with ethyl acetate (20 mL), extracted (2×brine, 20 mL), dried over Na₂SO₄ and evaporated to dryness to give an olive solid. The crude product is purified by flash chromatography (10 g silica gel, ETOAC-hexanes gradient 0%->80% ETOAC, flow 15 mL min⁻¹) followed by crystallization from ether/hexanes to afford the title compound as beige powder (21 mg, 33%), HPLC: t_{R}=5.8 min (system 1); ESI+MS: m/z=317.5 (MH⁺).

The starting material can be prepared as described hereafter:

6-(4-Chloro-phenylamino)-pyridazine-3-carboxylic acid

A solution of 6-chloropyridazine-3-carboxylic acid (0.5 g, 3.15 mmol, [5096-73-1]) and 4-chloroaniline (805 mg, 6.31 mmol) in 1,2-dimethoxyethane (5 mL) is microwave heated for 20 min at 100° C. After cooling the reaction mixture is diluted with ethyl acetate (10 mL) and stirred for 5 min. The brown precipitate is filtered off and triturated with cold water (30 mL). The light brown suspension is filtered and washed with water. After vacuum dry at 45° C. the product is obtained as a beige powder (250 mg, 32%).

Following the same procedure, the following compounds can be obtained:

### EXAMPLE 8.2

### rac-[6-(4-Chloro-phenylamino)-pyridazin-3-yl]-(3-methyl-piperidin-1-yl)-methanone

Gray powder, HPLC: t_{R}=6.1 min (system 1); ESI+MS: m/z=331.6 (MH⁺).

### EXAMPLE 8.3

### [6-(4-Chloro-phenylamino)-pyridazin-3-yl]-(3,3-dimethyl-piperidin-1-yl)-methanone

Beige powder, HPLC: t_{R}=6.3 min (system 1); ESI+MS: m/z=345.6 (MH⁺).

### EXAMPLE 8.4

### [6-(4-Chloro-phenylamino)-pyridazin-3-yl]-(3,4-dihydro-1H-isoquinolin-2-yl)-methanone

Beige powder, HPLC: t_{R}=6.4 min (system 1); ESI+MS: m/z=365.6 (MH⁺).

### EXAMPLE 8.5

### [6-(4-Chloro-phenylamino)-pyridazin-3-yl]-(4-methyl-piperidin-1-yl)-methanone

Gray powder, HPLC: t_{R}=6.1 min (system 1); ESI+MS: m/z=331.6 (MH⁺).

### EXAMPLE 9.1

### [5-Methyl-6-(6-methyl-pyridin-3-ylamino)-pyridin-3-yl]-piperidin-1-yl-methanone

To a solution of 5-methyl-6-(6-methyl-pyridin-3-ylamino)-nicotinic acid (130 mg, 0.534 mmol) in DMF (15 mL), HOBt (106 mg, 0.74 mmol) and 4-methylmorpholine (180 μL, 1.61 mmol) were added. After 10 min of stirring, EDC (146 mg, 0.74 mmol) and piperidine (74.6 μL, 0.74 mmol) were added and the resulting mixture was stirred at 50° C. for 16 hours. The solvent was removed in vacuo and EtOAc was added. The organic phase was washed with a saturated solution of NaHCO₃, dried over sodium sulfate and concentrated in vacuo to afford a brown resin. The crude product was purified by flash chromatography over silica gel using EtOAc as solvent to afford [5-methyl-6-(6-methyl-pyridin-3-ylamino)-pyridin-3-yl]-piperidin-1-yl-methanone (30 mg, 18%) as a yellow resin.

The starting material was prepared as described hereafter:

i) N-tert-butyldimethylsilyl isopropyl formimidate

At −40° C., to a suspension of isopropyl formimidate hydrochloride (12.9 g, 105 mmol) in DCM (150 mL), triethylamine (32.3 mL, 231 mmol) was added in once. Then, a solution of tert-butyldimethylsilyl triflate (24.6 mL, 105 mmol) in DCM (100 mL) was added drop-wise with keeping the temperature below −40° C. At the end of the addition, 25 mL of hexanes was added at once and the mixture was then allowed to reach RT. The precipitate was filtered off and washed with hexanes and DCM. The filtrate was concentrated in vacuo to afford a yellow paste. Et₂O was added and the residual triethylammonium triflate was removed by decantation. The ethereal phase was concentrated in vacuo to afford N-tert-butyldimethylsilyl isopropyl formimidate as a clear oil (15.53 g, 73.5%) which will be used without further purification.

ii) 6-Hydroxy-5-methyl-nicotinic acid ethyl ester

At RT, a solution of propionyl chloride (1.55 mL, 17.4 mmol) in 3.5 mL of toluene was added drop-wise to a solution of N-tert-butyldimethylsilyl isopropyl formimidate (3.51 g, 17.4 mmol) and triethylamine (12.2 mL, 87 mmol) in 10 mL of toluene. The resulting mixture was stirred at RT for 2 hours and then 10 mL of hexanes was added. The precipitate was removed by filtration and washed with hexanes (3×5 mL). The solution was concentrated in vacuo to afford a clear oil. This oil was solubilised in toluene (15 mL) and ethyl propiolate (1.2 mL, 11.6 mmol) was added. The resulting mixture was stirred at 85° C. for 70 hours. The mixture was concentrated in vacuo and then diluted with HCl 2N. The aqueous phase was extracted with DCM. The organic phases were combined, dried over sodium sulfate and concentrated in vacuo to afford a crude yellow paste (3.5 g). The crude product was purified by flash chromatography over silica gel using Hexanes/EtOAc (75/25 to 0/100) as solvent gradient to afford 6-hydroxy-5-methyl-nicotinic acid ethyl ester (1.65 g, 78.5%) as a yellow powder. (ES-MS: m/z 182.1 [M+H]⁺, t_{R} 3.28 min (system 2)).

iii) 6-Chloro-5-methyl-nicotinic acid ethyl ester

A mixture of 6-hydroxy-5-methyl-nicotinic acid ethyl ester (1.65 g, 9.11 mmol) in POCl₃ (2.55 mL, 27.3 mmol) was stirred at 120° C. for 1.5 hour. The mixture was cooled down and poured into ice. The resulting precipitate was filtered off, washed with water and then solubilised in DCM. The organic phase was dried over sodium sulfate and then concentrated in vacuo to afford 6-chloro-5-methyl-nicotinic acid ethyl ester (1.55 g, 85%) as a dark brown solid. (ES-MS: m/z 241.1/243.1 [M+CH₃CN+H]⁺, t_{R} 5.12 min (system 2)).

iv) 5-Methyl-6-(6-methyl-pyridin-3-ylamino)-nicotinic acid ethyl ester

A mixture of 6-chloro-5-methyl-nicotinic acid ethyl ester (750 mg, 3.76 mmol), 3-amino-6-methylpyridin (609 mg, 5.64 mmol), Pd(OAc)₂ (26 mg, 0.11 mmol), rac-BINAP (72 mg, 0.11 mmol) and potassium carbonate (2.62 g, 18.8 mmol) in degassed toluene (20 mL) was stirred, under argon, at 80° C. for 4 hours. EtOAc was added and the organic phase was washed with water, dried over sodium sulfate and concentrated in vacuo to afford the 5-methyl-6-(6-methyl-pyridin-3-ylamino)-nicotinic acid ethyl ester (1.02 g, 100%) as a black solid. (ES-MS: m/z 272.2 [M+H]⁺, t_{R} 3.37 min (system 2)).

v) 5-Methyl-6-(6-methyl-pyridin-3-ylamino)-nicotinic acid

To a solution of 5-methyl-6-(6-methyl-pyridin-3-ylamino)-nicotinic acid ethyl ester (1.02 g, 3.76 mmol) in THF/MeOH (1/1, 40 mL), NaOH 2N (3.8 mL, 7.6 mmol) was added. The mixture was stirred at RT for 16 hours. The solvent was removed in vacuo and the crude was diluted with water. The aqueous was acidified to pH 4-5 by addition of HCl 2N. The resulting precipitate was removed by filtration and dried under high-vacuum to afford 5-methyl-6-(6-methyl-pyridin-3-ylamino)-nicotinic acid (615 mg, 67%) as a beige solid. (ES-MS: m/z 244.1 [M+H]⁺, t_{R} 2.77 min (system 2)).

### EXAMPLE 9.2

### [5-Fluoro-6-(6-methyl-pyridin-3-ylamino)-pyridin-3-yl]-piperidin-1-yl-methanone

[5-Fluoro-6-(6-methyl-pyridin-3-ylamino)-pyridin-3-yl]-piperidin-1-yl-methanone was prepared following the procedure described in example 9.1.

TLC: R_{f}=0.14 (EtOAc/hexanes 1:9), LC/MS: m/z=315 (MH⁺).

The starting material can be prepared as described in example 9.1.v) and iv) starting from 6-Chloro-5-fluoro-nicotinic acid methyl ester.

### EXAMPLE 10

### Biological Testing

Activity of compounds of the present invention was examined by measurement of the inhibition of the glutamate induced elevation of intracellular Ca²⁺-concentration following similar methods than those described in L. P. Daggett et al., Neuropharm. Vol. 34, pages 871-886 (1995), P. J. Flor et al., J. Neurochem. Vol. 67, pages 58-63 (1996).

The table below represents percentages of inhibition of the glutamate induced elevation of intracellular Ca²⁺-concentration at a concentration of 10 μM.

| Compound | mGluR5 Activity |
|---|---|
| Number | inh. at 10 μM [%] |
| 1.1 | 95 |
| 1.2 | 93 |
| 1.3 | 75 |
| 1.4 | 37 |
| 1.5 | 90 |
| 1.6 | 96 |
| 1.7 | 77 |
| 1.8 | 54 |
| 1.9 | 97 |
| 1.10 | 94 |
| 1.11 | 71 |
| 1.12 | 93 |
| 1.12a | 99 |
| 1.12b | 100 |
| 1.13 | 96 |
| 1.14 | 94 |
| 1.15 | 88 |
| 1.16 | 54 |
| 1.17 | 83 |
| 1.18 | 32 |
| 1.19 | 94 |
| 1.20 | 95 |
| 1.21 | 57 |
| 2.1 | 60 |
| 2.2 | 46 |
| 2.3 | 65 |
| 2.4 | 100 |
| 2.5 | 97 |
| 2.6 | 98 |
| 2.7 | 98 |
| 2.8 | 96 |
| 2.9 | 100 |
| 2.10 | 98 |
| 2.10a | 99 |
| 2.10b | 99 |
| 2.11 | 100 |
| 2.12 | 100 |
| 2.13 | 96 |
| 2.14 | 97 |
| 2.15 | 100 |
| 2.16 | 97 |
| 2.17 | 97 |
| 2.18 | 100 |
| 2.19 | 96 |
| 2.20 | 96 |
| 2.21 | 98 |
| 2.22 | 98 |
| 2.23 | 100 |
| 2.24 | 97 |
| 3.1 | 94 |
| 3.2 | 97 |
| 3.3 | 90 |
| 4.1 | 99 |
| 4.2 | 97 |
| 4.3 | 92 |
| 4.4 | 98 |
| 4.5 | 88 |
| 4.6 | 86 |
| 4.7 | 87 |
| 4.8 | 94 |
| 4.9 | 37 |
| 4.10 | — |
| 4.11 | 51 |
| 4.12 | 96 |
| 4.13 | 97 |
| 4.13a | 97 |
| 4.13b | 96 |
| 4.14 | 86 |
| 4.15 | 98 |
| 4.16 | 81 |
| 4.17 | 98 |
| 4.18 | 97 |
| 4.19 | 97 |
| 4.20 | 100 |
| 4.21 | 99 |
| 4.22 | 98 |
| 4.23 | 95 |
| 4.24 | 92 |
| 4.25 | 98 |
| 4.26 | 93 |
| 4.27 | — |
| 4.28 | 93 |
| 4.29 | 86 |
| 4.30 | 96 |
| 4.31 | 100 |
| 4.32 | 100 |
| 4.33 | 96 |
| 4.34 | 98 |
| 4.35 | 85 |
| 4.36 | — |
| 4.37 | 94 |
| 4.38 | 98 |
| 4.39 | 96 |
| 4.40 | 98 |
| 4.41 | 96 |
| 4.42 | 98 |
| 4.43 | 100 |
| 4.44 | 99 |
| 4.45 | 90 |
| 4.46 | 94 |
| 4.47 | 97 |
| 4.48 | 99 |
| 4.49 | 66 |
| 4.50 | 98 |
| 4.51 | 96 |
| 4.52 | 96 |
| 4.53 | 100 |
| 4.54 | 97 |
| 4.55 | 96 |
| 4.56 | 97 |
| 4.57 | 97 |
| 4.58 | 93 |
| 4.59 | — |
| 4.60 | 97 |
| 4.61 | 79 |
| 4.62 | 99 |
| 4.63 | 100 |
| 4.64 | 99 |
| 4.65 | 83 |
| 4.66 | 91 |
| 4.67 | 95 |
| 4.68 | 99 |
| 4.69 | 99 |
| 4.70 | 96 |
| 4.71 | 99 |
| 4.72 | 97 |
| 4.73 | 72 |
| 4.74 | 92 |
| 4.75 | 96 |
| 4.76 | 92 |
| 4.77 | 100 |
| 5.1 | 98 |
| 5.2 | 100 |
| 5.2a | 100 |
| 5.2b | 98 |
| 5.3 | 87 |
| 5.4 | 83 |
| 5.5 | 96 |
| 6.1 | 97 |
| 6.2 | 100 |
| 6.3 | 95 |
| 6.4 | 98 |
| 6.5 | 33 |
| 6.6 | 54 |
| 6.7 | 35 |
| 7.1 | 98 |
| 8.1 | 91 |
| 8.2 | 100 |
| 8.3 | 82 |
| 8.4 | 89 |
| 8.5 | 86 |
| 9.1 | 97 |
| 9.2 | 99 |

1. A compound of formula (I) 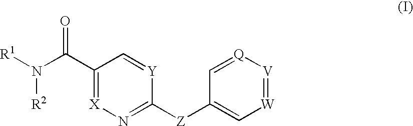
wherein
R¹ represents optionally substituted Alkyl or optionally substituted Benzyl and
R² represents Hydrogen (H, optionally substituted Alkyl or optionally substituted Benzyl; or
R¹ and R² form together with the Nitrogen atom to which they are attached an optionally substituted heterocycle with less than 14 ring atoms;
R³ represents Halogen, Hydroxy (OH), Alkyl, Alkoxy, Amino, Alkylamino, Dialkylamino;
R⁴ represents Hydroxy (OH), Halogen, Amino, Alkylamino, Dialkylamino Alkyl, Alkoxy;
Q represents CH, CR⁴, N;
V represents CH, CR⁴, N;
W represents CH, CR⁴, N;
X represents CH; N;
Y represents CH, CR³, N;
Z represents CR^{6a}R^{6b}, NR⁵, O;
R⁵ represents Hydrogen, Hydroxy (OH);
R^{6a} and R^{6b} are each independently selected from Hydrogen, Halogen, Hydroxy (OH), Amino, Alkyl, Alkoxy, Haloalkyl; and
provided that Q, V, W are not N at the same time, in free base or acid addition salt form.

2. The compound according to claim 1 of formula (I), 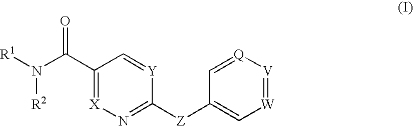
wherein
Q represents CH, CR⁴, N;
V represents CH, CR⁴, N;
W represents CH, CR⁴, N;
X represents CH, N;
Y represents CH, CR³, N;
Z represents CR^{6a}R^{6b}, NR⁵, O;
R¹ represents optionally substituted Alkyl or optionally substituted Benzyl and
R² represents Hydrogen (H) optionally substituted Alkyl or optionally substituted Benzyl; or
R¹ and R² form together with the Nitrogen atom to which they are attached an optionally substituted heterocycle with less than 14 ring atoms;
R³ represents Halogen, Hydroxy (OH), Alkyl, Alkoxy, Amino, Alkylamino, Dialkylamino;
R⁴ represents Hydroxy (OH), Halogen, Amino, Alkylamino, Dialkylamino Alkyl, Alkoxy;
R⁵ represents Hydrogen, Hydroxy (OH);
R^{6a} and R^{6b} are each independently selected from Hydrogen, Halogen, Hydroxy (OH), Amino, Alkyl, Alkoxy, Haloalkyl; and
provided that Q, V, W are not N at the same time and provided that at least one Q, V, W represents N, in free base or acid addition salt form.

3. The compound according to claim 1 represented by formula (I), 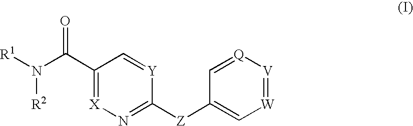
wherein
Q represents CH, CR⁴, N;
V represents CH, CR⁴, N;
W represents CH, CR⁴, N;
X represents CH, N;
Y represents CR³;
Z represents CR^{6a}R^{6b}, NR⁵, O;
R¹ represents optionally substituted Alkyl or optionally substituted Benzyl and
R² represents Hydrogen (H) optionally substituted Alkyl or optionally substituted Benzyl; or
R¹ and R² form together with the Nitrogen atom to which they are attached an optionally substituted heterocycle with less than 14 ring atoms;
R³ represents Halogen, Hydroxy (OH), Alkyl, Alkoxy, Amino, Alkylamino, Dialkylamino;
R⁴ represents Hydroxy (OH), Halogen, Amino, Alkylamino, Dialkylamino Alkyl, Alkoxy;
R⁵ represents Hydrogen, Hydroxy (OH);
R^{6a} and R^{6b} are each independently selected from Hydrogen, Halogen, Hydroxy (OH), Amino, Alkyl, Alkoxy, Haloalkyl; and
provided that Q, V, W are not N at the same time and provided that at least one Q, V, W represents N, in free base or acid addition salt form.

4. The compound according to claim 1 having the formula (IV) 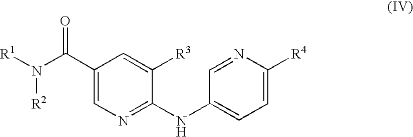
R¹ represents optionally substituted Alkyl or optionally substituted Benzyl and
R² represents Hydrogen (H), optionally substituted Alkyl or optionally substituted Benzyl; or
R¹ and R² form together with the Nitrogen atom to which they are attached an optionally substituted heterocycle with less than 14 ring atoms;
R³ represents Halogen, Alkyl, Alkoxy, Amino, Alkylamino, Dialkylamino; and
R⁴ represents Hydroxy (OH), Halogen, Alkyl, Alkoxy.

5. The compound of claim 1, selected from the group consisting of
6(4-Chloro-phenylamino)-N,N-diethyl-nicotinamide hydrochloride,
N,N-Diethyl-6-p-tolylamino-nicotinamide hydrochloride;
N,N-Diethyl-6-(4-methoxy-phenylamino)-nicotinamide hydrochloride,
6-(4-Chloro-phenylamino)-N,N-bis-(2-methoxy-ethyl)-nicotinamide hydrochloride;
[6-(4-Chloro-3-fluoro-phenylamino)-pyridin-3-yl]-piperidin-1-yl-methanone;
[6(4-Bromo-phenylamino)-pyridin-3-yl]-piperidin-1-yl-methanone;
4-[5-(Piperidine-1-carbonyl)-pyridin-2-ylamino]-benzonitrile;
Piperidin-1-yl-[6-(4-trifluoromethoxy-phenylamino)pyridin-3-yl]-methanone;
[6-(4-Chloro-phenylamino)-pyridin-3-yl]-(2-methyl-piperidin-1-yl)methanone hydrochloride;
(2-Methyl-piperidin-1-yl)-(6-p-tolylamino-pyridin-3-yl)-methanone;
[6-(4-Methoxy-phenylamino)-pyridin-3-yl]-(2-methyl-piperidin-1-yl)methanone hydrochloride;
rac-[6-(4-Chloro-phenylamino)-pyridin-3-yl]-(3-methyl-piperidin-1-yl)-methanone;
[6-(4-Chloro-phenylamino)-pyridin-3-yl]-(S-3-methyl-piperidin-1-yl)-methanone;
[6-(4-Chloro-phenylamino)-pyridin-3-yl]-(R-3-methyl-piperidin-1-yl)-methanone;
3-Methyl-piperidin-1-yl)-(6-p-tolylamino-pyridin-3-yl)methanone;
[6-(4-Methoxy-phenylamino)-pyridin-3-yl]-(3-methyl-piperidin-1-yl)-methanone hydrochloride;
(3-Methyl-piperidin-1-yl)-(6-phenylamino-pyridin-3-yl)methanone;
[6-(3-Chloro-phenylamino-pyridin-3-yl]-(3-methyl-piperidin-1-yl)-methanone hydrochloride;
[6(Chloro-phenylamino)-pyridin-3-yl]-morpholin-4-yl-methanone hydrochloride;
[6-(4-Methoxy-phenylamino)-pyridin-3-yl]-morpholin-4-yl)-methanone hydrochloride;
cis-[6-(4-Chloro-phenylamino)-pyridin-3-yl]-(2,6-dimethyl-morpholin-4-yl)-methanone hydrochloride;
(cis-2,6-Dimethyl-morpholin-4-yl)-(6-p-tolylamino-pyridin-3-yl)-methanone hydrochloride,
(cis-2,6-Dimethyl-morpholin-4-yl)-[6-(4-methoxy-phenylamino)-pyridin-3-yl]-methanone hydrochloride;
[6-(5-Chloro-pyridin-2-ylamino)-pyridin-3-yl]-piperidin-1-yl-methanone;
Azepan-1-yl-[6-(pyridin-3-ylamino)-pyridin-3-yl]-methanone;
[6-(3,4-Difluoro-phenylamino)-pyridin-3-yl]-piperidin-1-yl-methanone;
rac-(2-Aza-bicyclo[2.2.1]hept-2-yl)-[5-chloro-6-(4-chloro-phenylamino-pyridin-3-yl]-methanone;
[5-Chloro-6-(4-chloro-phenylamino)-pyridin-3-yl]-thiomorpholin-4-yl)-methanone;
rac-[5-Chloro-6-(6-methoxy-pyridin-3 ylamino)-pyridin-1-yl]-(3-methyl-piperidin-1-yl)-methanone
Azepan-1-yl-[5-chloro-6-(6-methoxy-pyridin-3-ylamino)-pyridin-3-yl]-methanone;
[5-Chloro-6-(methoxy-pyridin-3-ylamino)-pyridin-3-yl]-piperidin-1-yl-methanone
[5-Chloro-6-(6-ethoxy-pyridin-3-ylamino)-pyridin-3-yl]-piperidin-1-yl-methanone;
rac-[5-Chloro-6(6-methyl-pyridin-3-ylamino)-pyridin-3-yl]-(3-methyl-piperidin-1-yl)-methanone;
[5-Chloro(—S(methyl-pyridin-3-ylamino)-pyridin-3-yl]-(S-3-methyl-piperidin-1-yl-methanone;
[5-Chloro-6-(6-methyl-pyridin-3-ylamino)-pyridin-3-yl]-(R-3-methyl-piperidin-1-yl)-methanone;
[5-Chloro-6(6-methyl-pyridin-3-ylamino)-pyridin-3-yl]-piperidin-1-yl-methanone;
Azepan-1-yl-[5-chloro-6-(6-methyl-pyridin-ylamino)-pyridin-3-yl]-methanone;
rac-(2-Aza-bicyclo[2.2.1]hept-2-yl)-[5-chloro-6-(6-methyl-pyridin-3-ylamino)-pyridin-3-yl]-methanone;
[5-Chloro (6-methyl-pyridin-3-ylamino)pyridin-3-yl]-thiazolidin-3-yl-methanone;
[5-Chloro-6-(6-methyl-pyridin-3-ylamino)-pyridin-3-yl]-thiomorpholinyl-methanone;
[5-Chloro-6-(2-methyl-pyrimidin-5-ylamino)-pyridin-3-yl]-(3-methyl-piperidin-1-yl-methanone;
[5-Chloro-6-(2-methyl-pyrimidin-5-ylamino)pyridin-3-yl]-piperidin-1-yl)-methanone;
Azepan-1-yl-[5-chloro-6-(2-methyl-pyrimidin-5-ylamino)-pyridin-3-yl]-methanone;
[5-Chloro-6-(6-methyl-pyridin-3-ylamino)-pyridin-3-yl]-(3-ethyl-piperidin-1-yl)-methanone;
[5-Chloro-6-(6-methoxy-pyridin-3-ylamino)-pyridin-3-yl]-(3-ethyl-piperidin-1-yl)-methanone;
[5-Chloro-6-(4-chloro-phenylamino)-pyridin-3-yl]-(3-ethyl-piperidin-1-yl)-methanone;
[5-Chloro-6-(6-methyl-pyridin-3-ylamino)-pyridin-3-yl]-(3-propyl-piperidin-1-yl)-methanone;
[5-Chloro-6(6-trifluoromethyl-pyridin-3-ylamino)-pyridin-3-yl]-((R)-2-ethyl-piperidin-1-yl)-methanone;
[5-Chloro-6-(6-methoxy-pyridin-3-ylamino)-pyridin-3-yl]-((R)-2-ethyl-piperidin-1-yl)-methanone;
rac-[5-Chloro-6-(6-methyl-pyridin-3-yloxy)-pyridin-3-yl]-(3-methyl-piperidin-1-yl)-methanone;
[5-Chloro-(6-methyl-pyridin-3-yloxy)-pyridin-3-yl]-piperidin-1-yl-methanone;
Azepan-1-y[5-chloro-6(6-methyl-pyridin-3-yloxy)-pyridin-3-yl]-methanone;
[6-(6-Methyl-pyridin-3-ylamino)-pyridin-3-yl]-piperidin-1-yl-methanone;
Azepan-1-yl-[6-(4-chloro-phenylamino)-pyridin-3-yl]-methanone;
[6-(4-Chloro-phenylamino)-pyridin-3-yl]-(3,3-difluoro-piperidin-1-yl)-methanone;
[6-(4-Chloro-phenylamino)-pyridin-3-yl]-(4-methyl-piperidin-1-yl)-methanone;
[6(4-Chloro-phenylamino)-pyridin-3-yl]-(3,5-dimethyl-piperidin-1-yl)methanone;
rac-[6(4-Chloro-phenylamino)-pyridin-3-yl]-(3-hydroxymethyl-piperidin-1-yl)-methanone;
rac-[6-(4-Chloro-phenylamino)-pyridin-3-yl]-(3-methoxy-piperidin-1-yl)-methanone;
[6-(4-Chloro-phenylamino)-pyridin-3-yl](octahydro-quinolin-1-yl)-methanone (diastereomeric mixture, cis/trans);
(3-Aza-bicyclo[3.2.2]non-3-yl)-[6-(4-chloro-phenylamino)-pyridin-3-yl]-methanone;
(2-Aza-tricyclo[3.3.1.1*3,7]dec-2-yl)-[6(4-chloro-phenylamino)-pyridin-3-yl]-methanone;
[6-(4-Chloro-phenylamino)-pyridin-3-yl]-(3-hydroxy-8-aza-bicyclo[3.2.1]oct-8-yl)methanone;
rac-(2-Aza-bicyclo[2.2.1]hept-2-yl)-[6-(4-chloro-phenylamino)-pyridin-3-yl]-methanone;
rac-(3-Methyl-piperidin-1-yl)-[6-(6-methyl-pyridin-3-ylamino)-pyridin-3-yl]-methanone;
(S-3-Methyl-piperidin-1-yl)-[6-(6-methyl-pyridin-3-ylamino)-pyridin-3-yl]-methanone;
(R-3-Methyl-piperidin-1-yl)[6(6-methyl-pyridin-3-ylamino)pyridin-3-yl]-methanone;
[6-(4-Chloro-phenylamino)pyridin-3-yl]-(rel-(3aR,4S,7aR)-4-hydroxy-4-m-tolylethynyl-octahydro-indol-1-yl)-methanone;
Azepan-1-yl-[6-(6-methyl-pyridin-3-ylamino)-pyridin-3-yl]-methanone;
Azocan-1-yl-[6-(6-methyl-pyridin-3-ylamino pyridin-3-yl]-methanone;
[5-Chloro-6-(6-methyl-pyridin-3-ylamino)-pyridin-3-yl](2-ethyl-piperidin-1-yl)-methanone;
[5-Chloro-6(6-methyl-pyridin-3-ylamino)-pyridin-3-yl]-((R)-2-ethyl-piperidin-1-yl)-methanone;
[5-Chloro-6-(6-methyl-pyridin-3-ylamino)-pyridin-3-yl]-((S)-2-ethyl-piperidin-1-yl)-methanone;
[5-Chloro-6-(6-methyl-pyridin-3-ylamino)-pyridin-3-yl](2,3-dimethyl-piperidin-1-yl)-methanone;
[5-Chloro-6-(6-methyl-pyridin-3-ylamino)-pyridin-3-yl]((2S,3S)-2,3-dimethyl-piperidin-1-yl)-methanone;
[5-Chloro-6(6-methyl-pyridin-3-ylamino)-pyridin-3-yl]-((2R,3R)-2,3-dimethyl-piperidin-1-yl)-methanone;
[5-Chloro-6-(6-methyl-pyridin-3-ylamino)-pyridin-3-yl]-((S)-2-methyl-piperidin-1-yl)-methanone;
[5-Chloro-6-(6-methyl-pyridin-3-ylamino)-pyridin-3-yl]-((R)-2-methyl-piperidin-1-yl)-methanone;
[5-Chloro-6-(6-methyl-pyridin-3-ylamino)-pyridin-3-yl]-(octahydro-[1]pyridin-1-yl)-methanone;
[5-Chloro-6-(6-methyl-pyridin-3-ylamino)-pyridin-3-yl]-(4aS,7aS)-octahydro-[1]pyridin-1-yl-methanone;
[5-Chloro-6-(6-methyl-pyridin-3-ylamino)-pyridin-3-yl]-(4aR,7aR)-octahydro-[1]pyridin-1-yl-methanone;
[5-Chloro-6-(6-methyl-pyridin-3-ylamino)-pyridin-3-yl]-(2-isopropyl-piperidin-1-yl)methanone;
[Chloro-6-(6-methyl-pyridin-3-ylamino)-3-yl]-((R)-2-isopropyl-piperidin-1-yl)-methanone;
[5-Chloro-6-(6-methyl-pyridin-3-ylamino)pyridin-3-yl]((S)-2-isopropyl-piperidin-1-yl)-methanone;
[5-Chloro-6-(6-methyl-pyridin-3-ylamino)-pyridin-3-yl]-((R)-3-ethyl-piperidin-1-yl)-methanone;
[5-Chloro-(6-methyl-pyridin-3-ylamino)pyridin-3-yl]-((S)-3-ethyl-piperidin-1-yl)-methanone;
[5-Chloro-6-(6-methyl-pyridin-3-ylamino)-pyridin-3-yl](3-cyclopropyl-piperidin-1-yl)-methanone;
[5-Chloro-6-(6-methyl-pyridin-3-ylamino)-pyridin-3-yl]-(2-propyl-piperidin-1-yl)-methanone;
[5-Chloro-6-(6-methyl-pyridin-3-ylamino)-pyridin-3-yl]-((S)-2-propyl-piperidin-1-yl)-methanone;
[5-Chloro-6(6-methyl-pyridin-3-ylamino)-pyridin-3-yl]-((R)-2-propyl-piperidin-1-yl)-methanone;
[5 Chloro-6-(6-methyl-pyridin-3-ylamino)-pyridin-3-yl](2,3-diethyl-piperidin-1-yl)-methanone;
(2-Butyl-piperidin-1-yl)-[5-chloro-6-(6-methyl-pyridin-3-ylamino)-pyridin-3-yl]-methanone;
[5-Chloro-6(6-methyl-pyridin-3-ylamino)-pyridin-3-yl]-[2-(1-ethyl-propyl)-piperidin-1-yl]-methanone;
[5-Chloro-6(6-methyl-pyridin-3-ylamino)-pyridin-3-yl]-(2-ethyl-3-methyl-piperidin-1-yl)-methanone;
[5-Chloro-6(6-methyl-pyridin-3-ylamino)-pyridin-3-yl]-(2-phenyl-piperidin-1-yl)-methanone;
[5-Chloro-6(6-methyl-pyridin-3-ylamino)-pyridin-3-yl]-(3,4,5,6-tetrahydro-2H-[2,2′]bipyridinyl-1-yl)-methanone;
[5-Chloro-6-(6-methyl-pyridin-1-ylamino)-pyridin-3-yl]-(3,4,5,6-tetrahydro-2-[2,3′]bipyridinyl-1-yl)-methanone;
[5-Chloro-6-(6-methyl-pyridin-3-ylamino)-pyridin-3-yl]-[2-(tetrahydro-furan-2-yl)-piperidin-1-yl]-methanone;
[5-Chloro-6-(6-methyl-pyridin-3-ylamino)-pyridin-3-yl-(2-(6-methyl-furan-2-yl)-piperidin-1-yl]-methanone;
[5-Chloro-6-(6-methyl-pyridin-3-ylamino)-pyridin-3-yl]-(2-oxazol-2-yl)-piperidin-1-yl)-methanone;
[2-(2-Chloro-ethyl)-piperidin-1-yl]-[5-chloro-6-(6-methyl-pyridin-3-ylamino)-pyridin-3-yl]-methanone;
[5-Chloro-6(6-methyl-pyridin-3-ylamino)-pyridin-3-yl]-(2,6-dimethyl-piperidin-1-yl)-methanone;
[5-Chloro-6(6-methyl-pyridin-3-ylamino)-pyridin-3-yl]-(2,2,6,6-tetramethyl-piperidin-1-yl)-methanone;
[5-Chloro-6-(6-methyl-pyridin-3-ylamino)-pyridin-3-yl]-(2-methyl-6-propyl-piperidin-1-yl)-methanone;
[5-Chloro-6-(6-methyl-pyridin-3-ylamino)-pyridin-3-yl]-((2R,6R)-2-ethyl-6-propyl-piperidin-1-yl)-methanone;
[5-Chloro-6-(6-methyl-pyridin-3-ylamino)-pyridin-3-yl]-(5-methyl-2-propyl-piperidin-1-yl)-methanone;
[5-Chloro-6-(4-chloro-phenylamino)-pyridin-3-yl]-(octahydro-1]pyridin-1-yl)-methanone;
[5-Chloro-6-(4-chloro-phenylamino)-pyridin-3-yl]-((R)-2-ethyl-piperidin-1-yl)-methanone;
[5-Chloro-6-(6-methyl-pyridin-3-ylamino)-pyridin-3-yl]-(2-vinyl-piperidin-1-yl)-methanone;
[5-Chloro-6-(6-methyl-pyridin-3-ylamino)-pyridin-3-yl]-[((Z)-2-propenyl)-piperidin-1-yl]-methanone;
[5-Chloro-6-(6 methyl-pyridin-3-ylamino)-pyridin-3-yl]-[3-ethylidene-piperidin-1-yl]-methanone;
[5-Chloro-6-(6-methyl-pyridin-3-ylamino)-pyridin-3-yl]-[3-propylidene-piperidin-1-yl]-methanone;
[5-Chloro-6-(6-methyl-pyridin-3-ylamino)-pyridin-3-yl]-(2-ethoxymethyl-piperidin-1-yl)-methanone;
[5-Chloro-6(6-methyl-pyridin-3-ylamino)pyridin-3-yl]-(2-ethoxymethyl-piperidin-1-yl)-methanone;
[5-Chloro-6-(6-methyl-pyridin-3-ylamino)-pyridin-3-yl]-[2-(2-hydroxy-ethyl)-piperidin-1-yl]-methanone;
[5-Chloro-6-(6-methyl-pyridin-3-ylamino)pyridin-3-yl]-(5-fluoro-2-propyl-piperidin-1-yl)-methanone;
[5-Chloro-6-(6-methyl-pyridin-3-ylamino)-pyridin-3-yl]-[2-(1,2-difluoro-propyl)-piperidin-1-yl]-methanone;
[5-Chloro-6-(6-methyl-pyridin-3-ylamino)-pyridin-3-yl]-[2-(2-fluoro-propyl)-piperidin-1-yl]-methanone;
[5-Chloro-6-(6-methyl-pyridin-3-ylamino)-pyridin-3-yl]-(2-ethyl-[1,3]oxazepan-3-yl)-methanone;
[5-Chloro-6-(6-methyl-pyridin-3-ylamino)pyridin-3-yl]-[2-propyl-[1,3]oxazepan-3-yl)-methanone;
[5-Chloro-6-(6-methyl-pyridin-3-ylamino)-pyridin-3-yl]-2-(1-ethyl-propyl)-[1,3]oxazepan-3-yl)-methanone;
[5-Chloro-6-(6-methyl-pyridin-3-ylamino)-pyridin-3-yl]-(2-propyl-[1,3]oxazinan-3-yl)-methanone;
[5-Chloro-6-(6-methyl-pyridin-3-ylamino)pyridin-3-yl]-[2-(1-ethyl-propyl)-[1,3]oxazinan-3-yl]-methanone;
(2-Butyl-[1,3]oxazinan-3-yl)-[5-chloro-6(6-methyl-pyridin-3-ylamino)-pyridin-1-yl]-methanone;
[5-Chloro-6-(6-methyl-pyridin-ylamino)pyridin-3-yl]-[2-(2-methoxy-ethyl)-piperidin-1-yl]-methanone;
[5-Chloro(6-methyl-pyridin-3-ylamino)-pyridin-3-yl]-(2-phenyl-pyrrolidin-1-yl)-methanone;
[5-Chloro-6-(6-methylpyridin-3-ylamino)-pyridin-3-yl]-(2-pyridin-2-yl-pyrrolidin-1-yl)-methanone;
[5-Chloro-6-(6-methyl-pyridin-3-ylamino)-pyridin-3-yl]-((R)-2-ethoxy-pyrrolidin-1-yl)-methanone;
[5-Chloro-6-(6-methyl-pyridin-3-ylamino)-pyridin-3-yl]-[2-(5-methyl-thiophen-2-yl)-pyrrolidin-1-yl]-methanone;
[5-Chloro-(6-methyl-pyridin-3-ylamino)-pyridin-3-yl]-(2-propyl-azepan-1-yl)-methanone;
[5-Chloro-6-(6-methyl-pyridin-3-ylamino)-pyridin-3-yl]-(3-propyl-morpholinyl)-methanone;
rac-[5-Chloro-6-(4-chloro-phenylamino)pyridin-3-yl]-(3-methylpiperidin-1-yl)-methanone;
[5-Chloro-6-(4-chloro-phenylamino)-pyridin-3-yl]-(S-3-methyl-piperidin-1-yl)-methanone;
[5-Chloro-6-(4-chloro-phenylamino)-pyridin-3-yl(R-3-methyl-piperidin]-yl)-methanone;
Azepan-1-y[2-(4-chloro-phenylamino)-pyrimidin-5-yl]-methanone;
[2-(4-Chloro-phenylamino)-pyrimidin-5-yl]-piperidin-1-yl-methanone;
rac-[2-(4-Chloro-phenylamino)-pyrimidin-5-yl]-(3-methyl-piperidin-1-yl)-methanone;
Azepan-1-yl-[6-(4-chloro-phenylamino)-methoxy-pyridin-3-yl]-methanone,
Azepan-1-yl-[5-methoxy-6-(6-methylpyridin-3-ylamino)-pyridin-3-yl]-methanone];
[6-(4-Chloro-phenylamino)-5-methoxy-pyridin-3-yl]-piperidin-1-yl-methanone;
[5-Methoxy-6-(6-methyl-pyridin-3-ylamino)-pyridin-3-yl]-piperidin-1-yl-methanone;
Azepan-1-yl-[6(4-chloro-phenylamino)-5-ethoxy-pyridin-3-yl]methanone;
Azepan-1-yl-[5-ethoxy-6-(6-methyl-pyridin-3-ylamino)-pyridin-3-yl]-methanone;
[5-Ethoxy-6-(6-methyl-pyridin-3-ylamino)-pyridin-3-yl]-piperidin-1-yl-methanone;
[5-Chloro-6-(6-chloro-pyridin-3-ylamino-pyridin-3-yl]-(3-methyl-piperidin-1-yl)-methanone;
[6-(4-Chloro-phenylamino)-pyridazin-3-yl]-piperidin-1-yl-methanone;
rac-[6(4-Chloro-phenylamino)-pyridazin-3-yl]-(3-methyl-piperidin-1-yl)-methanone;
[6(4-Chloro-phenylamino)-pyridazin-3-yl]-(3,3-dimethyl-piperidin-1-yl)-methanone;
[6-(4-Chloro-phenylamino)-pyridazin-3-yl]-(3,4-dihydro-1H-isoquinolin-2-yl)-methanone;
[6(4-Chloro-phenylamino)-pyridazin-3-yl]-(4-methyl-piperidin-1-yl)-methanone;
[5-Methyl-6-(6-methyl-pyridin-3-ylamino)-pyridin-3-yl]-piperidin-1-yl-methanone; and
[5-Fluoro-6(6-methyl-pyridin-3-ylamino)-pyridin-3-yl]-piperidin-1-yl-methanone.

6. A process for the preparation of the compound of claim 1, or a salt thereof,
a) wherein Z represents NH or O, comprises the step of reacting a compound of formula (ii) 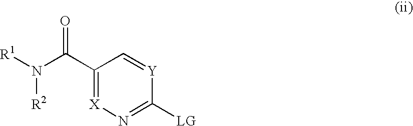
wherein R¹ and R² are as defined above, LG represents a leaving group with a compound of formula (iii) 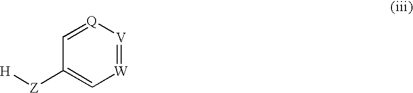
wherein Q, V, W is as defined above and Z represents NH or O,
and recovering the resulting compound of formula (I) in free base or acid addition salt form
or
b) wherein Z represents CH₂, comprises the step of reacting a compound of formula (ii) 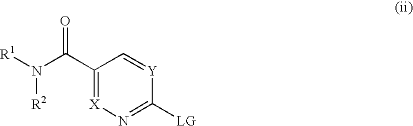
wherein R¹ and R² are as defined in claim 2, LG represents a leaving group, with a compound of formula (vi) 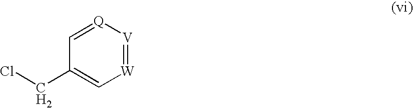
wherein Q, V, W is as defined above and Z, optionally in the presence of a reaction auxiliary, and recovering the resulting compound of formula (I) in free base or acid addition salt form.

7-8. (canceled)

9. A pharmaceutical composition, comprising:
the compound of claim 1 free base or pharmaceutically acceptable acid addition salt form, in association with a pharmaceutical carrier or diluent.

10-11. (canceled)

12. A method of the treatment of disorders associated with irregularities of the glutamatergic signal transmission, and nervous system disorders, and of GI, Urinary tract disorders mediated full or in part by mGluR5, comprising:
administering to a subject in need of such treatment a therapeutically effective amount of the compound of claim 1 in free base or pharmaceutically acceptable acid addition salt form.
